(19) United States
(12) Patent Application Publication
(10) Pub. No.: US 2026/0316074 A1
(43) Pub. Date: Oct. 15, 2026

(54) Microfluidic Devices Containing Micro-Immunobeads-Based Electromagnetic Operation Systems And Methods Of Use Thereof

(71) Applicants: The University of Hong Kong, Hong Kong (CN); ADVANCED BIOMEDICAL INSTRUMENTATION CENTRE LIMITED, Hong Kong (CN)

(72) Inventors: Xiaoxue FAN, Hong Kong (CN); Xinyu YE, Hong Kong (CN); Chuen Chung CHANG, Hong Kong (CN); Ho Cheung SHUM, Hong Kong (CN); Haisong LIN, Hong Kong (CN)

(21) Appl. No.: 19/569,782

(22) Filed: Mar. 17, 2026

Related U.S. Application Data
(60) Provisional application No. 63/786,606, filed on Apr. 10, 2025.

(51) Int. Cl. B01L 3/00

(52) U.S. Cl. CPC B01L 3/502715; B01L 3/502761; B01L 2200/025; B01L 2200/028; B01L 2200/0647; B01L 2300/12

(57) ABSTRACT

Microfluidic devices and methods for automated analyte detection are provided. The microfluidic devices contain a biochip and an electromagnetic (EM) chip with a programmable navigation matrix. The biochip includes a sample loading chamber, one or more wash chambers, a binding chamber, a detection chamber, and optionally a stop buffer chamber, all of which may be in fluid communication with each other. Immunomagnetic exosome beads can be pre-loaded or added separately to capture target analytes. The EM chip contains electromagnetic coils, switching circuits, magnetic motors, and a microcontroller that activates the coils to manipulate the beads. The coils are aligned with the chambers in the biochip. Optionally, the device includes an optical module for signal detection and an operation module for signal processing.

### CROSS-REFERENCE TO RELATED APPLICATIONS

This application claims the benefit of and priority to U.S. Provisional Application No. 63/786,606 filed Apr. 10, 2025, which is herein incorporated by reference in its entirety.

### FIELD OF THE INVENTION

The disclosed invention is generally in the field of microfluidic devices and specifically in the area of automated microfluidic devices for analyte detection.

### BACKGROUND OF THE INVENTION

The growing trend of the aging population has brought new challenges to global health and social systems. This demographic shift necessitates preparedness in public health, particularly in addressing age-related and chronic diseases that require timely diagnosis and management, such as neurological disorders, cardiovascular diseases, and metabolic syndromes^{1,2}. These conditions often progress silently over time and can significantly impair quality of life if not detected early³⁻⁵. With the continued expansion of the elderly population, the incidence and prevalence of such diseases are expected to rise markedly, placing an increasing burden on healthcare systems, caregivers, and society as a whole^{2,3,6-8}. Consequently, there is a growing need for early, accessible, and automated diagnostic tools to facilitate timely interventions and large-scale screening programs⁹⁻¹².

Current diagnostic standards for many chronic conditions rely on clinical evaluations¹³⁻¹⁵ and advanced imaging techniques¹⁶⁻¹⁸, which can suffer from a lack of objectivity in assessment¹⁹⁻²¹ and limited accessibility for broader implementation^{16,18,22}. Invasive molecular assays, such as those involving cerebrospinal fluid sampling or tissue biopsies, are often unsuitable for routine or early-stage screening due to associated risks and discomfort^{23,24}. To address these limitations, blood-based testing has emerged as a potential direction in diagnostics, offering a minimally invasive, scalable solution for detecting a wide range of analytes associated with chronic and age-related conditions^{3,25-28}.

Despite existing efforts, challenges remain in the development of effective blood tests for disease-related biomarkers. For example, the use of specific proteins, exosomal contents, or molecular markers as analytes in plasma or serum is still under evaluation, with inconsistent findings across prior studies^{29,3}^{⁰}. In some cases, the lack of clear correlation between circulating biomarker levels and clinical or imaging outcomes limits the utility of these markers in definitive diagnosis³¹⁻³³. In addition, there is a shortage of automated, user-friendly platforms that can carry out blood-based assays efficiently and at scale^{34,35}. Conventional tools like the enzyme-linked immunosorbent assay (ELISA), though widely used, are often time-intensive, labor-dependent, and require skilled personnel due to their multistep manual procedures³⁶. Alternative technologies such as electrochemical sensors (e.g., field-effect transistors)³⁷ and optical methods (e.g., Surface-enhanced Raman spectroscopy)³⁸ demand specialized equipment and tightly controlled conditions, reducing their feasibility for decentralized or point-of-care applications³⁹.

Therefore, systems and devices are needed for rapid, and accurate detection of clinically relevant analytes, while also supporting automation, scalability, and ease of use across a variety of healthcare and point-of-care settings.

It is an object of the invention to provide microfluidic devices for the detection of an analyte in a biological sample.

It is another object of the invention to provide automated microfluidic devices for the detection of an analyte in a biological sample.

It is another object of the invention to provide methods of using the microfluidic devices for the detection of an analyte in a biological sample.

### SUMMARY OF THE INVENTION

Microfluidic devices and systems thereof for automated analyte detection are provided. The microfluidic device includes (a) a biochip, and (b) an electromagnetic chip containing a programmable navigation matrix. Generally, the biochip and the electromagnetic chip are oriented parallel to each other, and the electromagnetic chip is directly below the biochip. Optionally, the microfluidic device further includes (c) an optical module for detecting a signal generated in the detection chamber, and (d) an operation module for processing the detected signal.

The biochip includes (i) a sample loading chamber; (ii) one or more wash chambers; (iii) a binding chamber; (iv) a detection chamber, and optionally (v) a stop buffer chamber. Optionally, the sample loading chamber, one or more wash chambers, the binding chamber, and the detection chamber are in fluid communication with each other. Optionally, the plurality of immunomagnetic exosome beads is provided as a separate component or is pre-loaded in the sample loading chamber.

The electromagnetic chip includes a programmable navigation matrix containing a plurality of electromagnetic coils, one or more switch integrated circuits electrically connected to the plurality of electromagnetic coils, one or more magnetic motors, and a microcontroller configured to regulate the activation of electromagnetic coils for electromagnetic actuation of the magnetic motor. The electromagnetic coils are arranged such that they align with the sample loading chamber, the one or more wash chambers, the binding chamber, and the detection chamber. The electromagnetic chip can further include a material exchange area that is in fluid connection with the sample loading chamber, the one or more wash chambers, the binding chamber, and the detection chamber.

Also provided are methods of detecting an analyte in a biological sample using the microfluidic device described herein. An exemplary method includes the following steps: (a) loading the biological sample into the sample loading chamber of the biochip of the microfluidic device; and (b) initiating the generation of an electromagnetic field.

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A-1J show schematic illustration of the MEMOs. FIG. 1A shows a schematic overview of using MEMOs for Alzheimer's disease (AD) diagnosis by detecting Exo-Aβ42 in plasma. FIG. 1B shows an exploded-view illustration of the main components of the MEMOs, which includes a sample processing microfluidic exosome detection biological chip (μ-Exo Biochip), a microbead actuation electromagnetic chip (μ-Bead EMchip), and an optical signal detection module. FIG. 1C is an optical image of a representative MEMOs operating in the sample loading state. Scar bar: One Hong Kong dollar coin. FIG. 1D is a schematic showing that the μ-Exo Biochip incorporates an iME beads-based Exo-Aβ42 identification assay (iME beads and reagents) designed for the detection of Exo-Aβ42. FIG. 1E shows the mechanism of the iME beads-based Exo-Aβ42 identification assay on the μ-Exo Biochip, which involves first identifying the exosome membrane endogenous protein CD63, subsequently recognizing the exosome membrane bound exogenous protein Aβ42, and finally generating signals. FIG. 1F shows the μ-Bead EMchip in MEMOs facilitates automated sample processing, which involves both droplet operation and bead operation. Inset: The step-motion mechanism of a magnetic motor, which begins with a programmed current applied to a specific coil, followed by the induction of a magnetic field, and ultimately driving the motor in the direction of the induced magnetic field. FIG. 1G shows the overall workflow of the μ-Bead EMchip for automation control. The yellow array indicates the programmed navigation paths of the magnetic motor to execute various bead and droplet motion commands. FIG. 1H is the cross-sectional illustration showing the interface between the integrated μ-Exo Biochip and μ-Bead EMchip. The magnetic motor is controlled by the μ-Bead EMchip through programmed magnetic field induction, triggering the motions of beads and droplets in the μ-Exo Biochip to complete the iME beads-based Exo-Aβ42 identification assay procedure. FIG. 1I is an overall illustration of the integrated system capable of performing diverse functionalities, including bead mixing, bead extraction, droplet merging and readout. FIG. 1J shows the optical readouts of the Exo-Aβ42 signal detected by MEMOs, which were utilized to distinguish Alzheimer's disease (AD), mild cognitive impairment (MCI), and healthy controls (CON).

FIGS. 2A-2M shows characterization of the capture efficiency and sensing performance of the iME beads-based Exo-Aβ42 identification assay. FIG. 2A is a schematic illustration of the functionalized immune magnetic exosome beads (iME beads). Anti-CD63 primary antibody (pink) is coated at the surface of the beads. FIG. 2B is a flow cytometry dot-plot analysis of control beads (left) and iME beads (right). APC dye is used to target the antibody for signaling. FIG. 2C is a histogram of the APC signal intensities for blank beads and iME beads. FIG. 2D is an SEM-EDS analysis elemental mapping (Fe—green, O—red, Au—blue) of iME beads. Gold nanoparticles (AuNPs) are conjugated to the antibody for visualization. Scale bar=1 μm. FIG. 2E shows the process of identifying and capturing the exosome membrane endogenous protein CD63 in the iME beads-based Exo-Aβ42 identification assay. FIG. 2F is a flow cytometry dot-plot analysis of single blank iME beads (left) and beads captured with exosomes (right). FITC dye is used to label exosomes. FIG. 2G is a histogram of the FITC signal intensities of the blank iME beads and the beads captured with exosomes. Error bars indicate SDs (n=3). FIG. 2H is a fluorescent microscope image of iME beads captured with FITC labeled exosomes (after extraction). Scale bar=10 μm. FIG. 2I shows the process of recognizing and signaling the exosome membrane bound exogenous protein Aβ42 in the iME beads-based Exo-Aβ42 identification assay. FIG. 2J shows improvement of the iME beads exosome capture quantity as a function of incubation time. Error bars indicate SDs (n=3). FIG. 2K shows improvement of iME beads exosome capture quantity as a function of the concentration of primary antibody. Error bars indicate SDs (n=3). FIG. 2L shows the specificity of the iME beads-based Exo-Aβ42 identification assay, showing the sensing signals of the target Exo-Aβ42 protein and three non-target proteins (Aβ42, CD63, and BSA). The baseline indicates the background signal of the optical detection sensor. FIG. 2M is a calibration plot of the response of the iME beads-based Exo-Aβ42 identification assay with 0-50 pg ml⁻¹ Exo-Aβ42 (Four-parameter logistic fitting, R²=0.987). Error bars indicate SDs (n=3). The baseline indicates the background signal of the optical detection sensor.

FIGS. 3A-3H show automation and validation of developed assay on chip. FIG. 3A is a schematic of the layer design of an exemplary μ-Exo Biochip. FIG. 3B shows simulated (lines) and experimental (dots) operation zones for beads and droplets based on adjusted bead loading mass and programmed magnet motor speeds. FIG. 3C are sequential optical images depicting bead operation (extraction of beads, Δt=150 ms, top) and droplet operation (transportation of droplets, Δt=500 ms, bottom). Scale bar=2 mm. FIG. 3D are sequential optical images showcasing the automation of the iME beads-based Exo-Aβ42 identification assay on-chip. The bottom section features a zoomed-in view of the actuation details, while the top right corner depicts the corresponding assay step. FIG. 3E shows animal samples tested on-chip (Alzheimer's mouse model: 5×FAD and wild-type mouse control: C57BL/6J). The 5×FAD Alzheimer's mouse model is transgenic for five AD-linked gene mutations. FIG. 3F is immunohistochemical (IHC) staining of Aβ42 aggregates in the hippocampus of C57BL/6J (top) and 5×FAD (bottom) mice. Dark plaques indicate Aβ42 deposits. FIGS. 3G and 3H are plots showing validation of the detection performance of MEMOs for sensing Exo-Aβ42 levels in AD mice (n=10) and wild-type mice plasma (n=10). Box plots (FIG. 3G) and waterfall plots (FIG. 3H) showing the optical signal readout by MEMOs. Statistical analysis was done using unpaired Mann-Whitney nonparametric test. *P<0.05, ** <0.01, *** P<0.00, ****P<0.0001.

FIGS. 4A-4G show validation of the clinical feasibility of MEMOs. FIG. 4A is a schematic showing a demonstration of the automated MEMOs detection workflow. FIG. 4B shows the conventional manual method workflow. FIG. 4C shows the corresponding distribution of response signals for 60 clinical samples, highlighting the changing trends associated with different stages of AD (n=20), MCI (n=10), and CON (n=30), as tested by the automated MEMOs detection system (AD, Alzheimer's disease patients; MCI, mild cognitive impairment patients; CON, healthy controls). FIG. 4D shows the corresponding distribution of the response signals for the same 60 clinical samples, illustrating the changing trends at various stages of AD (n=20), MCI (n=10), and CON (n=30), as tested by the conventional manual method. FIG. 4E are circle plots displaying the MEMOs sensing signal values of 60 clinical samples. FIG. 4F shows statistical significance analysis of the MEMOs sensing signals in AD, MCI and CON clinical plasma samples. Statistical analysis was done using unpaired Mann-Whitney nonparametric test. *P<0.05, ** <0.01, *** P<0.00, ****P<0.0001. FIG. 4G shows the receiver operating characteristic (ROC) curve analysis of 60 clinical samples based on MEMOs tested plasma Exo-Aβ42 levels.

FIGS. 5A-5G show development and evaluation of iME beads. FIG. 5A shows the functionalization process of the immune magnetic exosome beads (iME beads). FIGS. 5B-5G is flow cytometer characterization of the functionalization process of iME beads. Flow cytometry dot-plot analysis of blank beads (FIG. 5B), iME beads (FIG. 5C), blank beads with APC antibody dye (FIG. 5D), and iME beads with APC antibody dye (FIG. 5E), showing no cross-reaction between blank beads and APC antibody dye. Histogram (FIG. 5F) and mean (FIG. 5G) of the APC signal intensities of b, c, d, and e (n=3).

FIGS. 6A-6C show characterization of extracellular vesicles derived by SH-SY5Y neuronal cells. FIG. 6A is a Transmission electron microscope image of extracellular vesicles precipitated from SH-SH5Y cells. Scale bar: 100 nm. FIG. 6B is a graph showing size distribution of the isolated extracellular vesicles determined by nanoparticle tracking analysis, showing a mean value around 150 nm. FIG. 6C is a western blotting image of the isolated extracellular vesicles for exosomal markers (CD63 and TSG-101).

FIGS. 7A-7E shows flow cytometer characterization of exosome capture performance of iME beads. FIGS. 7A-7C are flow cytometry dot-plot analysis of blank iME beads (FIG. 7A), iME beads with exosome FITC dye (FIG. 7B), and iME beads captured with FITC labelled exosome (FIG. 7C), showing no cross-reaction between iME beads and exosome FITC dye. FIGS. 7D and 7E, Histogram (FIG. 7D) and mean (FIG. 7E) of the APC signal intensities of a, b, and c (n=3).

FIGS. 8A-8C shows characterization of the prepared Exo-Aβ42 proteins. FIG. 8A is a schematic illustration of the preparation process. FIG. 8B is a Transmission electron micrograph images of assembled Aβ42 aggregates (Left) and exosome-Aβ42 protein associations (Right). FIG. 8C are Scanning transmission electron microscopy images of AuNPs (10 nm) immune labelled Aβ42 aggregates (Left) and exosome-Aβ42 protein associations (Right). Exo-Aβ42 are labeled with AuNPs (10 nm) via a Aβ42-specific antibody. The nanoparticles appear as block dots.

FIG. 9 is an Exosome quantitation standard curve (n=3). Four-parameter logistic fitting, R²=0.996.

FIGS. 10A-10E show improvement of iME beads-based Exo-Aβ42 identification assay on chip. Signal to noise ratio of blocking buffer (FIG. 10A), washing buffer (FIG. 10B), wash time (FIG. 10C), HRP-conjugated anti-Aβ42 antibody concentration (FIG. 10D), and signal enhancer adding (FIG. 10E).

FIGS. 11A-11C show the corresponding calibration process of MEMOs. FIG. 11A is a schematic illustration of the corresponding calibration process of MEMOs. FIG. 11B is a commercial Aβ42 protein kit standard curve (n=3). Four-parameter logistic fitting, R²=0.999. FIG. 11C shows the reference signals of the intermediate process. iME beads captured Exo-Aβ42 markers are lysis and quantified by a commercial Aβ42 kit to characterize the level of Aβ42 that are bounded with exosomes (n=3).

FIGS. 12A-12C shows administration and implementation of μ-EMchip manipulation at software and hardware levels. FIGS. 12A-12C are simplified illustration of integrated programmed microcontroller (FIG. 12A), which is programmed by different predetermined motion commands (FIG. 12B) to activate the magnetic motors and achieve required operations at defined zones (FIG. 12C).

FIG. 13 is a graph showing characterization of optical sensor readout versus standard microplate reader readout. Error bars indicate SDs (n=3).

FIGS. 14A-14C shows characterization of plasma total Aβ42 protein levels in animal samples, including Alzheimer's mice model: 5×FAD (n=10) and wild type mice model: C57BL/6J (n=10). FIG. 14A shows a commercial Aβ42 protein kit standard curve (n=3). Four-parameter logistic fitting, R²=0.979. FIGS. 14B and 14C are box plots and waterfall plots showing levels of total Aβ42 in plasma measured by ELISA.

FIG. 15 is a receiver operating characteristic (ROC) curve analysis of Exo-Aβ42 levels in plasma measured by MEMOs and Aβ42 levels in plasma tested by ELISA in animal samples. Alzheimer's mice model: 5×FAD (n=10) and wild type mice model: C57BL/6J (n=10).

FIGS. 16A and 16B show characterization of the CD63 protein levels in exosome of human samples. FIG. 16A is a commercial CD63 protein kit standard curve (n32 3). FIG. 16B shows CD63 protein levels in clinical samples, AD (n=10), MCI (n=10), and CON (n32 10). (AD, Alzheimer's disease patients; MCI, mild cognitive impairment patients; CON, healthy controls)).

FIGS. 17A-17C shows characterization of plasma total Aβ42 protein levels in human samples, including AD (n =20), MCI (n=10) and healthy control (n=30). FIG. 17A are waterfall plots showing levels of total Aβ42 in plasma measured by ELISA. FIG. 17B is a statistical significance analysis of the plasma total Aβ42 protein levels in AD, MCI and CON clinical plasma samples. FIG. 17C is a receiver operating characteristic (ROC) curve analysis of total Aβ42 levels in plasma measured by ELISA in clinical samples.

FIG. 18A is an exploded view of an exemplary microfluidic device. FIG. 18B is a top perspective view of an exemplary biochip showing the chambers of the biochip. FIG. 18C is an exploded perspective view of an exemplary biochip showing the layers of the biochip. FIGS. 18D and 18E is a top view and a bottom view of an exemplary electromagnetic chip respectively. FIG. 18F is a side view of an exemplary electromagnetic chip. FIG. 18G is a top perspective view of an exemplary optical sensing module.

FIGS. 19A and 19B are perspective views of an exemplary microfluidic device in the open configuration. FIGS. 19C and 19D are perspective views of an exemplary microfluidic device in the closed configuration.

### DETAILED DESCRIPTION OF THE INVENTION

The disclosed method and compositions can be understood more readily by reference to the following detailed description of particular embodiments and the Example included therein and to the Figures and their previous and following description.

### I. Micro-immunobeads-based Electromagnetic Operation System (MEMOs)

Disclosed are microfluidic devices and automated systems thereof (also referred to herein as “microfluidic systems”). The microfluidic devices and automated systems thereof are particularly suitable for detecting one or more analytes (e.g., Aβ42 protein) in a biological sample (such as a plasma/blood sample or a cerebrospinal fluid sample) obtained for a subject.

Although not intending to be limiting, the microfluidic devices and automated systems thereof can provide the following advantages over conventional microfluidic immunoassays. First, the microfluidic system integrates sample processing, biochemical analysis, and data transmission into a compact, portable format, making it highly adaptable for point-of-care or decentralized testing environments. As such, the microfluidic devices and systems thereof facilitate fully automated operation, allowing the entire assay process—from sample loading to result readout—to be carried out without manual intervention, thereby reducing user error and increasing consistency. Second, the microfluidic systems support dual-marker targeting, such as the simultaneous detection of a surface protein (e.g., on an exosome) and a disease-specific biomarker (e.g., Aβ42 protein), which can increase specificity and improve diagnostic accuracy. The microfluidic systems also incorporate programmable electromagnetic actuation, allowing for precise and responsive control of both magnetic bead clusters and fluid droplets during various assay steps. Additionally, the microfluidic systems are capable of detecting low concentrations of analytes, such as in the picogram-per-milliliter (pg/mL) range, from small sample volumes.

The microfluidic system generally includes: (a) a biochip and an (b) an electromagnetic chip (“EM chip”) containing a programmable navigation matrix. The biochip and the electromagnetic chip are typically oriented parallel to each other, with the electromagnetic chip positioned directly beneath the biochip to facilitate seamless interaction with the assay components within the biochip (i.e., fluid droplets such as the biological sample and buffers, and immunomagnetic beads). This vertical alignment allows the electromagnetic chip to generate precisely localized magnetic fields that can directly influence the position and movement of assay components in real time.

For example, during an immunoassay, a droplet containing immunomagnetic beads is transported across various chambers in the biochip. As the droplet reaches each chamber, the EM chip activates specific electromagnetic coils directly beneath the droplet's path in the biochip to generate localized magnetic fields. These fields attract and move the magnetic beads within the droplet, thereby facilitating targeted interactions with binding and capture agents, mixing with wash buffers, and precise positioning for detection. Because the EM chip is aligned directly below the biochip, the generated magnetic fields are tightly coupled to the spatial layout of the fluidic paths to facilitate accurate, contactless, and programmable manipulation of the assay workflow within the microfluidic system.

### A. Biochip

The microfluidic system contains a biochip (e.g., μ-Exo Biochip). The biochip contains one or more chambers for carrying out an immunoassay. In some forms, the biochip contains a sample loading chamber, one or more wash chambers, a binding chamber, and a detection chamber. The sample loading chamber, one or more wash chambers, the binding chamber, and the detection chamber are in fluid communication with each other, such that droplets and/or immunomagnetic beads can move from/to and in between the different chambers.

Each of the chambers can have any suitable shape and dimensions, as long as they can perform their respective designed functions, such as mixing, antigen binding, conducting chemical reactions, etc. In some forms, each of the chambers contains a mixing area and a flow region, where mixing takes place in the mixing area and the flow region provides a path for the droplet/fluid to flow into and out of the chamber.

Generally, the biochip is a stationary biochip, i.e., they do not require rotation to generate centrifugal force to drive fluid droplets throughout the biochip. Instead, the fluid droplets and immunomagnetic beads can be moved through the biochip via a magnetic mechanism generated by the electromagnetic chip described below.

The buffer agents and other reagents can be pre-loaded into the respective chambers of the biochip during fabrication. In some forms, this can be achieved by creating multiple access holes in the top layer of the biochip, allowing reagents to be manually introduced into their respective chambers prior to final sealing. Additionally, a separate access hole can be positioned above the sample loading chamber, to allow a user to manually load the biological sample at the time of use. In other forms, such as for mass manufacturing, reagent pre-loading can be performed using automated dispensing technologies, such as inkjet or piezoelectric dispensing systems, or robotic displacement pipetting, which allow for precise and efficient reagent deposition into designated chambers. Following reagent loading, the biochip can be sealed, with the sample loading hole either left open or covered by a thin, soft film that can be easily pierced or removed by the user at the time of sample introduction. This approach allows for the generation of fully or partially pre-loaded biochips.

The biochip can have dimensions less than or greater than that of a standard microtiter plate. The layout of microfluidic patterns in the biochip can be configured to support a variety of assay protocols. For example, in some forms, the dimensions of the biochip can be tailored to perform an immunomagnetic enrichment (iME) bead-based assays for the identification of protein biomarkers. In some forms, the height of the biochip ranges from about 2 mm to about 6 mm, such as about 3 mm, about 3.5 mm, or about 4 mm. In some forms, the width of the biochip ranges from about 20 mm to about 60 mm, such as about 30 mm, about 43 mm, or about 50 mm. In some forms, the length of the biochip ranges from about 20 mm to about 60 mm, such as about 30 mm, about 43 mm, or about 55 mm. In one exemplary form, the biochip has dimensions of about 43 mm×about 43 mm×about 3.5 mm (length×width×height).

The biochip can be formed of a variety of materials and in multiple configurations, depending on the assay. In some forms, the biochip may be formed from one or more layers of materials. In some forms, the biochip contains one or more barrier layers, one or more chamber layers, and optionally one or more adhesive layers. For example, the biochip contains a first barrier layer, a chamber layer, and a second barrier layer arranged in a stack, where the first barrier layer is attached to a first surface of the chamber layer via a first adhesive layer and the second barrier layer is attached to a second surface of the chamber layer via a second adhesive layer, where the first and second surfaces are opposite to each other. When assembled, the one or more chamber layers form one or more chambers and one or more flow paths within which the assay components and immunomagnetic beads can be transported.

The one or more chamber layers can include a top layer and a bottom layer, which together enclose the internal fluidic structure; thereby providing structural integrity. The one or more barrier layers may be formed from a variety of materials that are compatible with microfluidic devices and are suitable for optical access within the detection chamber. For example, the one or more barrier layers, such as the top layer and bottom layer can be formed from transparent polymer films such as polyethylene terephthalate (PET). Other suitable materials for forming the one or more barrier layers include but are not limited to polycarbonate, cyclic olefin copolymer, cyclic olefin polymer, poly(methyl methacrylate), polyethylene naphthalate (PEN), clarified polypropylene, thermoplastic polyurethane, fluoropolymers such as Cytop, and cellulose acetate films.

The one or more chamber layers are positioned between the top and bottom barrier layers and generally define the internal three-dimensional structure of the fluidic network, (e.g., the fluidic transport path) within the biochip. The one or more chamber layers can be formed from one or more substrates composed of materials suitable for microfluidic patterning and fluid control, such as polymers with low surface energy and high chemical resistance. For example, the material for forming the chamber layer can be selected to facilitate the smooth transport of immunomagnetic beads, and droplets containing analytes, and minimize nonspecific adsorption of assay agents and the biological sample. Suitable materials for forming one or more chamber layers include but are not limited to Polytetrafluoroethylene, DMS, cyclic olefin copolymer, cyclic olefin polymer, poly(methyl methacrylate), polycarbonate, clarified polypropylene, Fluorinated Ethylene Propylene, Polyethylene Naphthalate (PEN), and TPU.

The one or more chamber layers are generally patterned (e.g., via laser patterning or 3D printing) with features—such as cavities, channels, and openings—that form multiple fluidic chambers and flow paths when enclosed by the barrier layers. The fluidic chambers and flow paths in the one or more chamber layers support various functions of the biochip, including fluid loading, reagent handling, binding reactions, and signal detection.

The biochips can be fabricated using any suitable methods, using one or more suitable materials to form one or more layers. For example, the biochip can be fabricated from thermoplastic polymers including polyethylene, polypropylene, polycarbonate, polystyrene, polymethyl methacrylate (PMMA), polyethylene terephthalate (PET), cyclic olefin polymer (COP), and cyclic olefin copolymer (COC), and other polymers which are suitable to mass-production. Optionally, the biochips can be fabricated by CNC-machining of large plates (including those larger than SBS standard plates), and the blank plates for CNC-machining may be injected molded themselves. The biochips may also be made by injection molding of large plates (including those larger than SBS standard plates). Optionally, the biochips can be coated (either prior to, at an intermediate step during, or following assembly) to improve performance. A wide range of coatings and treatments can be applied. For example, exposing channels with 0.5% bovine serum albumin reduces the binding of proteins present in the sample to channel walls. Optionally, hydrophobic products such as PFC 502A (Cytonix, Beltsville, Md.) can be applied to channels and chambers, to minimize bubble formation. Similarly, the elastomeric or non-elastomeric membranes of pneumatic and mechanical valve structures can be coated or otherwise treated to provide improved sealing properties.

In some forms, the surface of the fluidic chambers and/or flow paths can be coated with one or more coatings to reduce non-specific binding, to allow the attachment of binding ligands, for biocompatibility and for flow resistance. For example, polymeric hydrogels and gel pads can be coated on the surface of the fluidic chambers and flow paths to increase adherence of biological molecules to the surface of the fluidic chambers. Polymeric hydrogels and gel pads that can be used as binding layers to adhere to surfaces biological molecules include, but are not limited to, proteins, peptides, oligonucleotides, polynucleotides, and larger nucleic acid fragments. For example, oligonucleotide probes can be bound to the surface of a continuous layer of the hydrogel, or to an array of gel pads. The gel pads can be conveniently produced as thin sheets or slabs, typically by depositing a solution of acrylamide monomer, a crosslinker such methylene bisacrylamide, and a catalyst such as N, N, N′, N′-tetramethylethylendiamine (TEMED) and an initiator such as ammonium persulfate for chemical polymerization, or 2,2-dimethoxy-2-phenyl-acetophone (DMPAP) for photopolymerization, in between two glass surfaces (e.g., glass plates or microscope slides) using a spacer to obtain the desired thickness of the polymeric gel. For example, the biochip can be configured for assays that involve the functionalization of solid substrate surfaces within the microfluidic device. In such configurations, specific regions of the solid substrate within the biochip can be coated with capture ligands or other binding molecules. Magnetic beads can then be employed not only as functional assay components but also as a means to actuate and transport sample droplets across the chip. The motion of the beads under an applied magnetic field can be harnessed to drive droplet movement toward designated surface-coated regions, thereby facilitating controlled interactions between analytes in the droplets and the immobilized ligands on the substrate. Alternatively, as demonstrated in the non-limiting examples, the surface of magnetic beads are coated with a ligand to minimize non-specific binding to the internal surfaces of the biochip rather than immobilizing the internal surface of the biochip with a ligand.

The biochip optionally contains one or more adhesive layers to bond and seal the one or more barrier layers and one or more chamber layers of the biochip together. The one or more adhesive layers can include double-sided adhesives and/or pressure-sensitive films. Suitable adhesives can be formed from acrylic, silicone, or epoxy formulations. Other adhesives that can be used to bond and seal the one or more barrier layers and one or more chamber layers of the biochip together include, but are not limited to, UV-curable adhesives such as Norland Optical Adhesive (NOA) series, Dymax 204-CTH-F, or Loctite 3491, and thermal adhesives such as 3 M DP420 epoxy, Loctite Ablestik 566K, or Henkel Technomelt TC series.

### 1. Sample Loading Chamber

The sample loading chamber of the biochip is configured to receive one or more fluid droplets of the biological sample, and a plurality of immunomagnetic exosome beads. Optionally, the plurality of immunomagnetic exosome beads is pre-loaded into the sample loading chamber. The sample loading chamber can be fluidly connected to the binding chamber (directly or indirectly) via a flow path or a microfluidic channel. For example, the sample loading chamber is in direct fluid communication with the binding chamber where a second binding agent binds to the target antigen, or the sample loading chamber is in direct fluid communication with a first wash chamber and/or the binding chamber.

The sample loading chamber can have any size and shape suitable for accommodating a biological sample and facilitating efficient mixing with immunomagnetic beads. In some forms, the chamber can be defined by a circular or oval depression patterned into the chamber layer of the biochip, such as through laser micromachining. In some forms, the dimensions of the sample loading chamber can be comparable to those of wells used in microtiter plates, offering compatibility with standard sample volumes. For example, the sample loading chamber can have a cylindrical shape with a flat bottom surface and a continuous sidewall. The sidewall of the sample loading chamber can be slightly tapered outward from bottom to top to facilitate sample loading. Additionally, the bottom surface of the sample loading chamber can include a flow region, such as a small port or opening that provides fluid communication with a flow path (such as a microfluidic channel) leading to a downstream chamber, e.g., a binding chamber or a wash chamber.

The dimensions and volume of the sample loading chamber may be selected based on the desired sample volume. For example, the sample loading chamber can have an area ranging from about 5 mm² to about 30 mm². For example, the area of the sample loading chamber can be about 18 mm². The chamber may have a diameter of 2-5 mm and a depth of 1-3 mm.

The diameter and/or depth of the sample loading chamber can be varied based on desired sample volume, flow dynamics, and compatibility with magnetic bead manipulation or optical detection features within the biochip. In some forms, the diameter of the sample loading chamber ranges from about 1 mm to about 8 mm. For example, the diameter of the sample loading chamber can range from about 1 mm to about 7 mm, from about 1 mm to about 5 mm, from about 1 mm to about 3 mm, such as about 2.5 mm, about 3 mm, about 4 mm, or about 5 mm. In some forms, the depth of the sample loading chamber ranges from about 1 mm to about 5 mm, such as about 1.5 mm, about 2 mm, about 3 mm, or about 4 mm. In one exemplary form, the sample loading chamber has a diameter of about 5 mm and a depth of about 3 mm.

In some forms, the sample loading chamber can include a removable or pierceable seal or septum over the top opening to maintain sterility prior to use. The sample loading chamber can also optionally include a hydrophobic coating on the interior surfaces to prevent adhesion of the sample and/or magnetic beads.

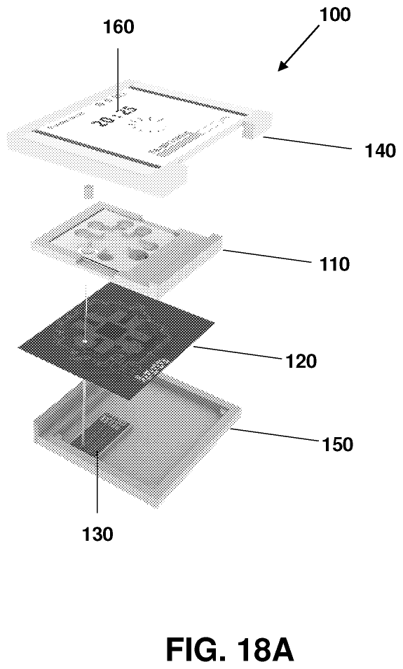

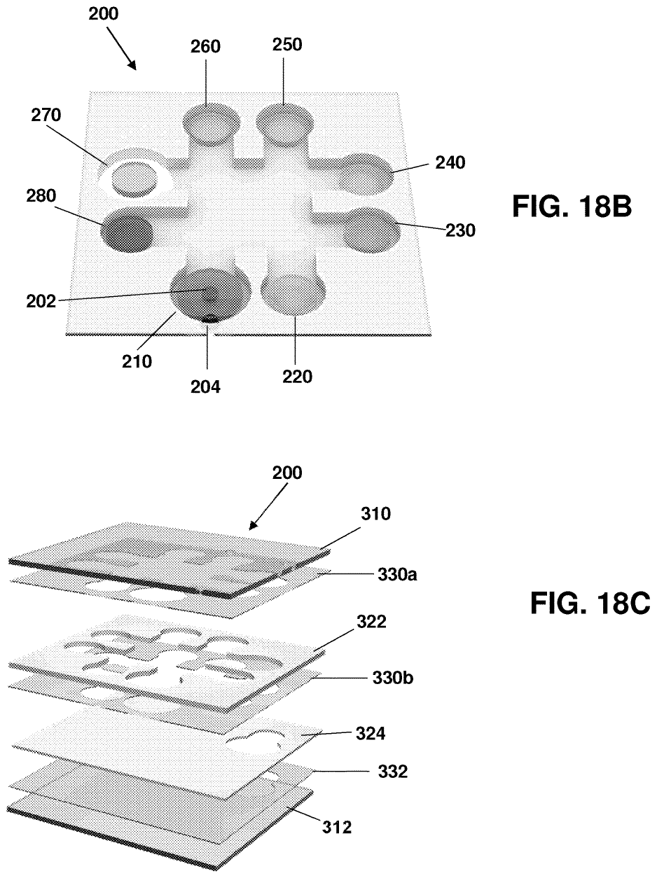

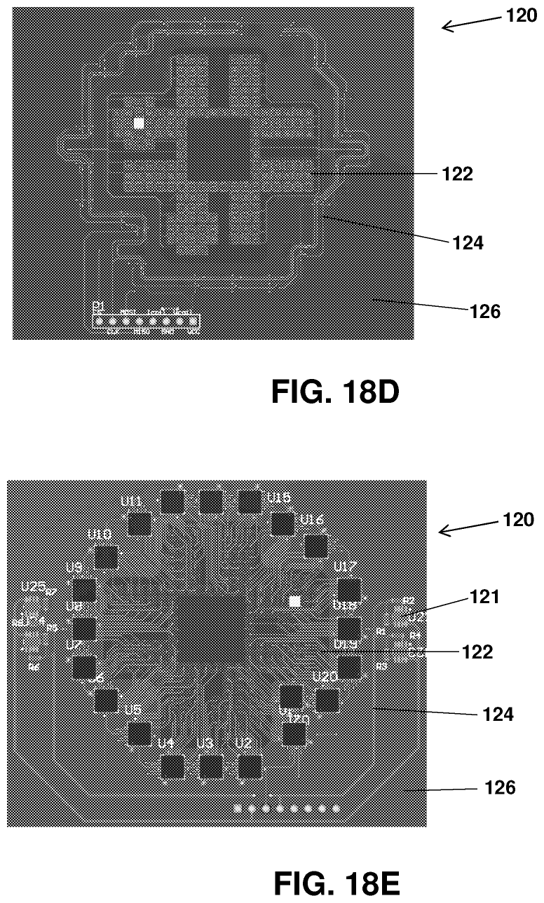

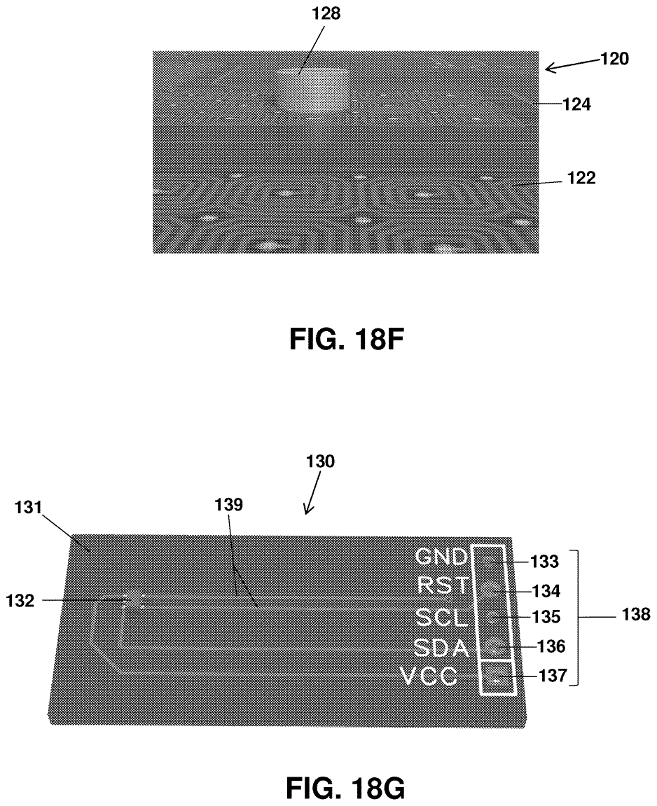

In some forms, the sample loading chamber includes a subchamber for housing pre-loaded immunomagnetic beads. For example, as illustrated in FIG. 18B, the sample loading chamber 210 can include a bead subchamber 202 configured to contain preloaded immunomagnetic beads. The bead subchamber 202 can be integrated within the sample loading chamber 210, allowing for immediate interaction between the sample and the beads upon loading. The biochip 200 can further include an access port or inlet 204 above the sample loading chamber 210, through which a user can manually introduce a biological sample, such as whole blood or plasma. Once introduced, the sample enters the sample loading chamber 210 and comes into contact with the preloaded immunomagnetic beads housed within the bead subchamber 202.

### 2. Wash Chambers

The biochip contains two or more wash chambers for sequential washing and reagent exchange steps during assay workflows. For example, the biochip can contain two, three, four, five, six, seven, eight, etc. wash chambers depending on the particular assay being performed on the biochip.

In some forms, the biochip contains four wash chambers. Optionally, a first wash chamber is arranged such that it is in direct fluid communication with the sample loading chamber and the binding chamber to facilitate the removal of unbound exosomes from the immunomagnetic beads. Optionally, a second wash chamber can be positioned in direct fluid communication with the binding chamber and the detection chamber to supply wash buffer after the binding step and prior to the detection step for the removal of unbound binding agents.

The wash chambers can have the same or different size and shape as the sample loading chamber described above. For example, each wash chamber can be circular or oval shaped, such as in the form of a well. Further, each wash chamber can have an area ranging from about 5 mm² to about 30 mm², such as about 18 mm².

In some forms, the diameter of the wash chamber ranges from about 1 mm to about 8 mm. For example, the diameter of the wash chamber can range from about 1 mm to about 7 mm, from about 1 mm to about 5 mm, from about 1 mm to about 3 mm, such as about 2.5 mm, about 3 mm, about 4 mm, or about 5 mm. In some forms, the depth of the wash chamber ranges from about 1 mm to about 5 mm, such as about 1.5 mm, about 2 mm, about 3 mm, or about 4 mm. In one exemplary form, the wash chamber has a diameter of about 4 mm and a depth of about 3 mm.

In some forms, the wash chamber is configured to house a preloaded buffer solution for washing, rinsing, or dilution steps. For example, as illustrated in FIG. 18B, the wash chamber 220, 240, 250, and/or 260, can contain one or more buffer solutions preloaded during assembly of the biochip. The biochip 200 can include fluidic pathways that direct droplets, such as those containing immunomagnetic beads with the analyte of interest, into the wash chamber, where they are mixed with the buffer solution to facilitate removal of unbound materials or to condition the sample for subsequent processing steps. Alternatively, the buffer solution is not preloaded into the wash chamber, but instead added at a later stage, such as prior to or during the assay process.

### 3. Binding Chamber

The biochip includes a binding chamber (e.g., an antibody chamber). The binding chamber is designed to expose the sample and/or immunomagnetic beads to a binding agent (e.g., Aβ42 antibodies) for the target antigen. The binding chamber is generally positioned along the fluid path that follows the sample loading and optionally an initial washing stages and precedes subsequent washing and detection steps positioned. For example, the binding chamber can be positioned between the first wash chamber and the second wash chamber.

The binding chamber can have the same size and shape as the other chambers on the on the biochip. For example, the binding chamber can have a circular geometry and can be formed as a depression or cavity within the chamber layer of the biochip. Alternatively, the binding chamber has a different size and shape from the other chambers on the biochip.

The binding chamber can have any dimension suitable for allowing sufficient volume for storing antibody reagents and facilitating interaction with bead-bound analytes. In some embodiments, the binding chamber has a diameter ranging from about 1 mm to about 7 mm, from about 1 mm to about 5 mm, from about 1 mm to about 3 mm, such as about 2.5 mm, about 3 mm, about 4 mm, or about 5 mm. Additionally, the depth of the binding chamber generally corresponds to the thickness of the chamber layer. In some embodiments, the binding chamber has a depth ranging from about 1 mm to about 5 mm, such as about 1.5 mm, about 2 mm, about 3 mm, or about 4 mm. In one exemplary form, the wash chamber has a diameter of about 4 mm and a depth of about 3 mm.

For example, similar to the sample loading chamber, the binding chamber can have a cylindrical shape with a flat bottom surface and a continuous sidewall. The sidewall of the antibody chamber can be slightly tapered outward from bottom to top to facilitate entry and exit of fluid droplets and/or bead clusters. Additionally, the bottom surface of the antibody chamber can include a flow region, such as a small port or opening, that provides fluid communication with a flow path, such as a microfluidic channel, leading to adjacent chambers, including an upstream chamber (e.g., the first wash chamber) and a downstream chamber (e.g., the second wash chamber).

In some forms, the binding chamber is configured to house a preloaded capture agent (e.g., antibodies) for use in target binding or detection steps. For example, as illustrated in FIG. 18B, the binding chamber 230 can contain a plurality of capture agents (such as a plurality of antibodies), preloaded during assembly of the biochip. The biochip 200 can include fluidic pathways that direct droplets, such as those containing the analyte of interest or immunomagnetic beads, into the binding chamber, where they are mixed with the capture agent to facilitate binding. Alternatively, the capture agent is not preloaded into the binding chamber, but instead added at a later stage, such as prior to or during the assay process, allowing for flexibility in capture agent selection or concentration based on specific assay requirements.

The binding agent suitable for use in the binding chamber can be selected depending on the assay and the analyte to be detected. In some forms, the binding agent can be an antibody, peptide, aptamer, or a nucleic acid aptamer. Typically, the binding agent in the binding chamber can bind the target antigen, but at a different region compared to the binding agent immobilized on the surface of the immunomagnetic beads. In other words, the first binding agent immobilized on the surface of the immunomagnetic beads and the second binding agent supplied in the binding chamber are typically an affinity pair that bind to the same target analyte.

In some forms, the one or more binding agents that are specific to a wide range of target analytes associated with exosomes. These binding agents can include, but are not limited to, antibodies, aptamers, peptide ligands, or nucleic acid probes designed to selectively recognize and bind to target proteins, peptides, or nucleic acids such as DNA and RNA. In some embodiments, binding agents can be selected to recognize surface markers characteristic of exosomes, facilitating detection and profiling based on cell origin or disease state. Other binding agents can be tailored to interact with membrane lipid components of exosomes, facilitating the identification of exosome subtype or function. Additionally, the biochip can incorporate binding agents specific to pathogen-derived antigens that become associated with exosomes following infection or immune activation. These can include ligands or antibodies targeting lipopolysaccharide (LPS), bacterial virulence factors, or viral proteins that are present in exosomes shed from infected host cells.

### 4. Detection Chamber

The biochip includes a detection chamber that acts as the reaction site for signal generation and optical detection of a signal from the sample. For example, the binding agent bound to the antigen in the binding chamber contains a reactive label, which reacts with a reactant supplied in the detection chamber and generates light and/or color for detection. Optionally, the detection chamber is in direct fluid communication with a stop buffer chamber, which allows delivery of the stop buffer droplet into the detection chamber following the reaction between the reactive label on the binding agent and the reactant.

The detection chamber generally has the same size and shape as the sample loading chamber and/or wash chambers. For example, the detection chamber can be a cylindrical or circular microfluidic chamber located within the chamber layer of the biochip. In some forms, the detection chamber includes a flat bottom surface and continuous sidewalls, which may be slightly tapered outward from bottom to top to support stable droplet confinement and facilitate controlled droplet merging and mixing. Alternatively, the detection chamber can be larger than the other chambers to accommodate larger volumes of fluid e.g., the fluid droplet from the sample and the stop buffer from the stop buffer chamber.

In some forms, the detection chamber is structurally distinct from other chambers in the biochip to facilitate accurate optical signal readout. For example, the detection chamber can be directly sealed between the top and bottom layers of the biochip, both of which can be formed from transparent materials that allow light to pass through without interference. In contrast, the intermediate layers of the biochip, can be fabricated from polytetrafluoroethylene (PTFE) or similar opaque materials, and can be hollowed out in the region corresponding to the detection chamber. This configuration prevents the opaque intermediate layers from obstructing the optical path, thereby facilitating reliable detection of light-based signals (e.g., fluorescence, absorbance) during assay readout.

Optionally, the floor of the detection chamber can be formed from an optically transparent material and is vertically aligned with one or more layers of the electromagnetic (EM) chip to allow unobstructed light transmission for signal readout. In some embodiments, the bottom surface of the detection chamber is precisely aligned with an optical sensor integrated into the bottom layer of the EM chip. To further facilitate optical detection, the substrate forming the bottom surface of the detection chamber can include a cutout or transparent region, to allow light to pass directly through the droplet and into the sensor without optical interference.

### 5. Stop Buffer Chamber

The biochip optionally includes a stop buffer chamber to receive an amount of a stop buffer for transfer to the detection chamber for stopping the reaction between the reactive label on the binding agent and the reactant. For example, the stop buffer chamber can be located adjacent to the detection chamber, to allow for direct transfer of the stop buffer droplet into the detection chamber.

Similar to the other chambers described above, the stop buffer chamber can be a cylindrical or circular microfluidic chamber formed within the chamber layer of the biochip. For example, the stop buffer chamber can include a flat bottom surface and a continuous sidewall, which may be slightly tapered outward from bottom to top to facilitate the entry and release of the stop buffer droplet.

### 6. Immunomagnetic Exosome Beads

The biochip can further include a plurality of immunomagnetic exosome beads pre-loaded into the sample loading chamber or provided as a separate component with the microfluidic system for loading at the time of performing an assay.

In some embodiments, the immunomagnetic beads are preloaded into the sample loading chamber or a portion thereof. For example, the immunomagnetic beads can be preloaded into a bead subchamber 202 within the sample loading chamber 210 (see FIG. 18B). In some forms, the biochip includes an access port or inlet 204 at the top surface of the biochip 200 through which a user can manually introduce a biological sample, such as whole blood or plasma (see FIG. 18B). Upon introduction, the sample directly enters the sample loading chamber 210 and mixes with the preloaded immunomagnetic beads contained in the bead subchamber 202.

In other embodiments, the immunomagnetic beads are not preloaded into the biochip but are instead introduced by the user during sample preparation. In such configurations, the user prepares a mixture of the biological sample (e.g., whole blood or plasma) and the immunomagnetic beads externally, prior to loading. This prepared mixture can then be manually introduced into the biochip through an access port or inlet above the sample loading chamber.

i. Magnetic Beads

The biochip includes a plurality of immunomagnetic beads. The immunomagnetic beads are typically functionalized to incorporate a first binding agent on the surface thereof for binding with the target analyte in a biological sample.

The magnetic beads can be any suitable beads formed from a magnetic material and can have any suitable shape. For example, the magnetic beads are microscale spherical particles which include a magnetic core embedded within or coated by a functionalizable outer layer. The core can be formed of superparamagnetic iron oxide nanoparticles (SPIONs), such as magnetite (Fe₃O₄) or maghemite (γ-Fe₂O₃).

Structurally, the beads are small enough to be manipulated through microfluidic channels without obstructing flow, yet large enough to be efficiently actuated by localized magnetic fields generated by the underlying electromagnetic chip. In some forms, the magnetic beads can range in diameter from about 5 to about 50 nanometers.

The outer surface of the beads is structurally modified or coated to include the binding agent, such as an antibody, an aptamer, a nanobody, a peptide, DNA/RNA, and/or other suitable ligands or chemical entities. The binding agent can be immobilized on the surface of the magnetic beads using any suitable coupling methods, such as through binding to a functional moieties on the surface of the magnetic beads, such as streptavidin, antibodies, or other ligands. The binding agent on the surface of the magnetic beads can have any suitable density. For example, the binding agent can be immobilized on the surface of the magnetic beads at high surface densities—e.g., in the range of 1-5 nmol of streptavidin per mg of beads—to facilitate interaction with target analytes.

In some forms, the magnetic beads can be incorporated into a polymer or inorganic matrix, forming bead structures with overall diameters in the range of 1 to 10 micrometers, for example, 2 to 5 micrometers. When a matric material is used, the matrix material forming the bead body can include a polymer, such as polystyrene, silica, and/or agarose, providing mechanical stability and chemical compatibility with assay reagents. In these forms, the binding agent can be immobilized on the surface of the magnetic beads or on the surface of the matrix, such as to the polymers and exposed on the surface of the matrix material.

In some forms, the immunomagnetic beads includes a functionalized outer layer containing the immobilized binding agent for targeted isolation of exosomes from the sample. Exemplary exosomal markers that can be used as the target of the binding agent include but is not limited to CD63, CD9, CD81, CD82, CD151, or a combination thereof. Additional exosomal markers that can be used as the target of the binding agent include but are not limited to heat shock proteins such as Anti-HSP70 and Anti-HSP90 (to capture HSP70 and HSP90 respectively), and phospholipids such as annexin V and Tim4 (to capture phosphatidylserine). For example, the binding agent used for exosome isolation can be an antibody, such as a CD63-specific antibody, which can be immobilized on the surface of the magnetic beads though any suitable coupling reaction. For example, the antibody is a biotinylated antibody, which can be immobilized onto the surface of streptavidin-coated magnetic beads through high-affinity biotin-streptavidin interactions. Once coated, the magnetic beads present specific surface-bound binding sites for exosome membrane proteins, allowing for the selective capture of exosomes from a sample. The binding agent can be an antibody, aptamer, nanobody, or other peptide.

The magnetic beads can be fabricated using a multi-step process that can include co-precipitation of magnetic nanoparticles, emulsification to form uniform spherical particles, and surface functionalization for biological compatibility and assay-specific functionality. The resulting beads should generally exhibit superparamagnetic behavior, with no residual magnetization in the absence of an external magnetic field, for allowing precise control and repositioning within the biochip.

In some forms, the binding agent is immobilized on the surface of the magnetic beads through non-covalent protein-ligand binding such as via a streptavidin-biotin interaction. In this configuration, the magnetic beads can be functionalized with streptavidin, while the binding agent is biotinylated, or vice versa. Alternatively, several other immobilization strategies can be employed depending on the desired assay configuration or surface chemistry.

One such alternative is passive adsorption, where binding agents, such as antibodies, adhere to the bead surface through non-covalent hydrophobic or electrostatic interactions. In some cases, magnetic beads can be pretreated with proteins such as protein A or protein G, which promote more efficient and oriented binding of antibodies through interaction with their Fc regions. Another approach includes covalent coupling, in which antibodies or other binding agents are chemically crosslinked to functionalized bead surfaces bearing reactive groups such as carboxyl, amine, or epoxy moieties. Exemplary chemistries used in covalent coupling include EDC/NHS-mediated crosslinking and glutaraldehyde-based conjugation.

Further, click chemistry can be used to achieve highly specific and efficient covalent immobilization through biorthogonal reactions, such as azide-alkyne cycloaddition, under mild and biocompatible conditions. Additionally, site-specific conjugation techniques—including Sortase-mediated ligation or HaloTag technology—can be used for selective control over the orientation and location of the binding agent on the bead surface.

Alternatively, the magnetic beads are commercially available. For example, non-coated or non-specifically coated magnetic microparticles based on polystyrene are available from Sigma-Aldrich (e.g., Catalog #49664, 39689, and 75597); streptavidin-coated magnetic beads such as those from Sigma-Aldrich (Catalog #08014 and 49532), and Dynabeads such as those from Invitrogen.

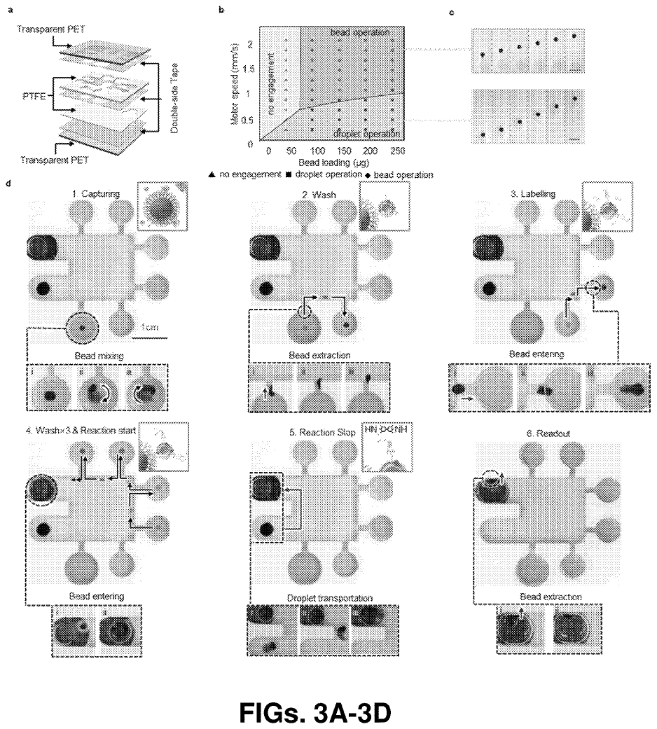

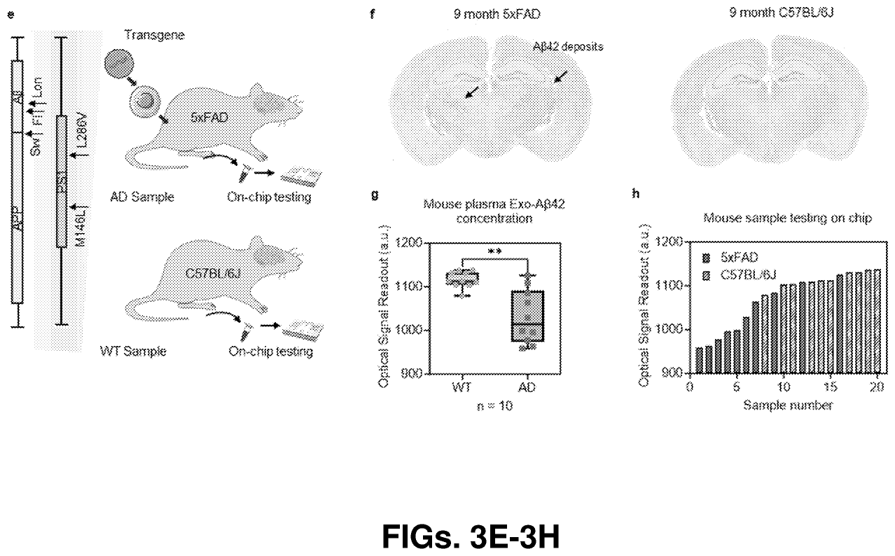

In some forms, the magnetic beads are configured to be removed from the detection chamber prior to detection of the analyte. For example, as shown in FIGS. 3B and 3C, magnetic beads can be extracted from individual droplets or used to transport droplets, depending on the loading mass of the beads and the speed of the magnetic motor. By carefully controlling these parameters, the system facilitates flexible droplet manipulation, allowing either bead-only extraction or bead-assisted droplet transport. In some forms, magnetic beads are first extracted from the sample loading chamber, where they have captured target molecules, thereby facilitating separation of the analyte from the sample droplet. The beads are then sequentially extracted through various processing chambers, including wash chamber 1, the antibody labeling chamber, wash chambers 2, 3, and 4, and ultimately into the detection chamber.

### 7. Other Biochip Components

i. Material Exchange Area And/or Channels

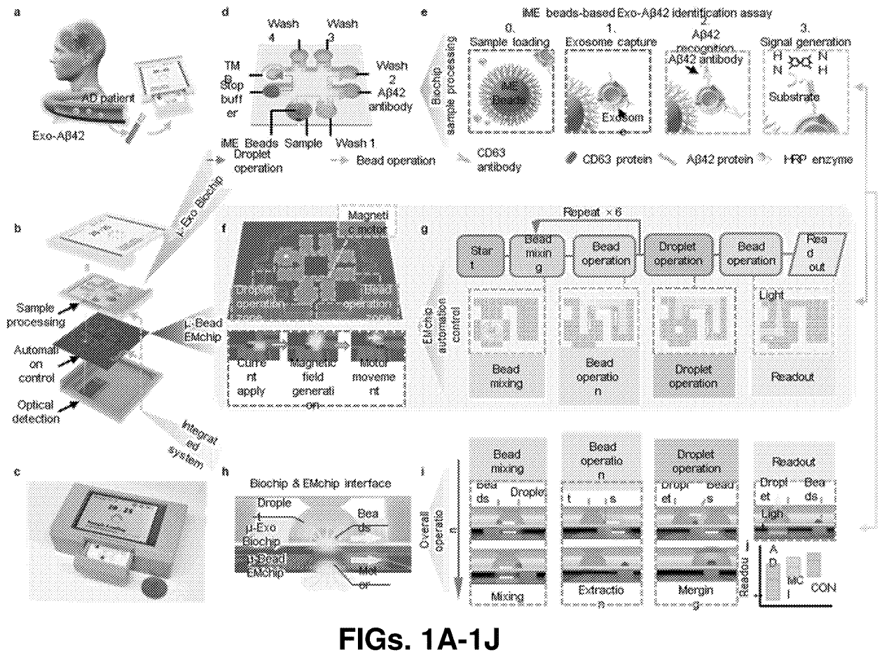

The biochip may further include a material exchange area or microfluidic channels in the chamber layer to facilitate flow of the sample and bead mixture between the sample loading chamber, the one or more wash chambers, the binding chamber, and the detection chamber. Optionally, when a material exchange area is included, the material exchange area is positioned centrally on the biochip (e.g., see FIG. 1D). Optionally, the material exchange area can include one or more microfluidic channels or conduits to facilitate the flow of the droplet from one chamber to another chamber (e.g., a lateral flow design).

In some forms, the material exchange area is configured as a central hub for the distribution and redirection of fluids within the biochip. For example, the material exchange area can be used to integrate samples with reagents and to facilitate the movement of these fluids into appropriate chambers for subsequent processing steps such as washing, binding, and detection. In some forms, the material exchange area is structured with the flow path and droplet chambers arranged around a central oil region. This configuration can minimize the water/oil interface, thereby reducing the risk of droplet contamination or mixing between adjacent reaction chambers. The central oil region serves as a physical and chemical barrier that separates the aqueous droplets loaded into different chambers, maintaining their integrity during processing and transport. Additionally, in the corresponding central area of the electromagnetic actuation chip (EMChip) described below, no coil patterns are implemented.

### 8. Exemplary Biochips

An exemplary biochip, the μ-Exo Biochip, is illustrated in FIGS. 18B and 18C. The μ-Exo Biochip 200 contains multiple functional chambers, including a sample loading chamber 210, wash chambers 220, 240, 250, 260, a binding chamber 230, a detection chamber 270, and a stop buffer chamber 280, all in fluid communication with one another via predefined microfluidic flow paths. The μ-Exo Biochip can be formed as a stationary platform, where movement of droplets and magnetic bead clusters is actuated by localized magnetic fields generated by an underlying electromagnetic chip.

As shown in FIG. 18C, the μ-Exo Biochip 200 is formed of multiple stacked layers, including two barrier layers 310, 312, two chamber layers 322, 324, and three adhesive layers 330a, 330b, 332. The two barrier layers, the top barrier layer 310 and the bottom barrier layer 312 form the top and bottom surfaces of the biochip 200. The top and bottom barrier layer can be formed from transparent materials such as polyethylene terephthalate (PET) to facilitate optical detection through the top barrier layer of the μ-Exo Biochip 200.

The two chamber layers 322, 324 of the μ-Exo Biochip 200 can be formed from polytetrafluoroethylene (PTFE) and includes a first chamber layer and a second chamber layer. As shown in FIG. 18C, the first chamber layer 322 can be patterned—such as by laser micromachining—to define the fluidic chambers and flow paths. The surface of the second chamber layer 324 can be used to form the floor of the chambers and the biochip, upon which, the assay agents and the sample will move along during use.

Three adhesive layers are used to adhere the two barrier layers 310, 312 and the two chamber layers 322, 324 of the μ-Exo Biochip together and seal the fluidic chambers and flow paths while maintaining flow access between chambers. As shown in FIG. 18C, a first adhesive layer 330a can be used to adhere the top barrier layer 310 to the first chamber layer 322, a second adhesive layer 330b can be used to adhere the first chamber layer 322 to the second chamber layer 324, and a third adhesive layer 332 can be used to adhere the second chamber layer 324 to the bottom barrier layer 312. The first and second adhesive layers 330a, 330b can be patterned similarly to the first chamber layer 322 to define the fluidic chambers and flow paths to facilitate non-adhesiveness of the chamber floors formed by the second chamber layer 324.

As shown in FIG. 18B, each chamber in the μ-Exo Biochip 200 can have a cylindrical geometry with a flat bottom surface and slightly tapered sidewalls. The sample loading chamber 210 is configured to receive the biological sample and immunomagnetic beads and includes a flow region that connects to downstream chambers. The sample loading chamber 210 can have a diameter of approximately 2 to 5 mm and a depth of 1 to 3 mm, sufficient to accommodate sample volumes of about 5 μL to about 200 μL. In some forms, the sample loading chamber have dimensions sufficient to accommodate sample volumes of about 5 μL to about 150 μL, about 5 μL to about 100 μL, about 5 μL to about 80 μL, about 5 μL to about 60 μL, about 5μL to about 40μL, about 5μL to about 20μL, or 5 μL to about 10 μL.

The μ-Exo Biochip includes a binding chamber 230 positioned between the first wash chamber 220 and the second wash chamber 240.

The μ-Exo Biochip can include four wash chambers, 220, 240, 250, and 260. As shown in FIG. 18B, the first wash chamber 220 is in direct fluid communication with the sample loading chamber 210 and binding chamber 230. For example, the first wash chamber 220 can be positioned between the sample loading chamber and adjacent to the binding chamber to allow for sequential washing steps to remove unbound material. As shown in FIG. 18B, the second, third, and fourth wash chambers 240, 250, 260, are positioned between the binding chamber 230 and detection chamber 270. Although three wash chambers are positioned between the binding chamber and detection chamber, any number of wash chambers can be included between the binding chamber and the detection chamber, such as one or two wash chambers.

As shown in FIG. 18B, the μ-Exo Biochip includes a detection chamber 270, such as a reaction chamber. The detection chamber 270 is aligned with an underlying optical sensor 130 (see FIG. 18A) can include a transparent bottom surface to allow light to pass through the droplet for absorbance-based signal readout.

The exemplary μ-Exo Biochip also includes a stop buffer chamber 280 adjacent to the detection chamber 270, which stores a droplet of stop solution that merges with the reactive droplet in the detection chamber 270 to stop the enzymatic reaction for signal development.

### B. Electromagnetic Chip

The microfluidic device also includes an electromagnetic chip that contains a programmable navigation matrix.

The electromagnetic chip contains a solid substrate upon which, a plurality of electromagnetic coils, one or more switch integrated circuits electrically connected to the plurality of electromagnetic coils, one or more magnetic motors, and a microcontroller configured to regulate the activation of electromagnetic coils for electromagnetic actuation of the magnetic motor. In some forms, the magnetic motor used for actuation in the biochip system is a permanent magnet, such as a neodymium magnet, which is formed of an alloy of neodymium, iron, and boron (NdFeB). Alternative permanent magnets can also be used, such as iron oxide-based magnets (e.g., Fe₃O₄ or γ-Fe₂O₃), cobalt (Co) magnets, nickel (Ni) magnets, and samarium-cobalt (SmCo) magnets.

Generally the plurality of the electromagnetic coils in the electromagnetic chip are arranged such that they align with the sample loading chamber, the one or more wash chambers, the binding chamber, the detection chamber, and if present, the stop buffer chamber.

The solid substrate of the electromagnetic (EM) chip can be formed from any suitable material capable of supporting the formation of electrical circuits and components. In some forms, the solid substrate contains a four-layer printed circuit board (PCB) structure, which facilitates the compact and functional integration of electromagnetic coils, magnetic motors, control circuitry, and electrical interconnections. The multilayer PCB structure can include at least two conductive and insulating layers, such as a first layer and a second layer. The first layer can be configured to house the magnetic motor, providing structural support and alignment for its movement across the chip. The second layer can be configured to embed the electromagnetic coil elements, allowing for the generation of localized magnetic fields for actuation of the magnetic motor. Optionally, the PCB structure includes a third and fourth layer, which can be used for mounting integrated circuits (ICs) and managing signal routing and power distribution.

Each layer of the EM chip can be formed of alternating conductive materials, such as copper, and dielectric insulating materials, such as FR-4, a fiberglass-reinforced epoxy laminate known for its mechanical strength, thermal stability, and electrical insulation. Depending on the application requirements, the EM chip can also be fabricated using other suitable substrate materials, including polyimide, PTFE-based laminates, or high-frequency-compatible composites, all of which provide improved electrical performance and environmental durability.

The solid substrate can be of any appropriate thickness, e.g., between about 0.1 and about 0.5 mm, between about 0.5 mm and about 1.0 mm, between about 1.0 and about 2.0 mm, between about 2.0 mm and about 3.0 mm, between about 3.0 mm and about 5.0 mm, such as between about 0.7 mm and about 1.0 mm. In some forms, the EM chip is constructed using a 4-layer PCB with a thickness ranging from about 0.6 mm to about 6 mm. Since magnetic flux density decreases with distance, a thinner PCB is preferred to improve magnetic driving capability and increase heat dissipation.

### 1. Electromagnetic Coils

The electromagnetic chip includes one or more electromagnetic coils structurally integrated, such as mounted on or embedding within a solid substrate. The electromagnetic coils can be arranged in a defined array or matrix configuration, aligned with specific regions or chambers of the biochip, including but not limited to the sample loading chamber, wash chambers, binding chamber, detection chamber, and if present, the stop buffer chamber. This spatial alignment supports targeted magnetic field application at predefined locations within the biochip.

Each electromagnetic coil can be formed from conductive wire, such as copper, wound around a core, which may be include a magnetic material (e.g., ferrite) to increase magnetic field strength. The dimensions of the coils—including inner diameter, number of turns, and spacing—are selected to achieve desired magnetic characteristics suitable for different microfluidic applications. The following are non-limiting considerations for the arrangement of the coils when preparing the EM chip.

Coil Shape: The coil can be designed to be square rather than circular because the wires that are perpendicular to the direction of the magnet's movement have the highest driving efficiency. The geometry of a square coil allows for more wire segments to be perpendicular to the magnetic field direction during the magnet's motion, thus improving the interaction efficiency with the magnetic field.

Coil Dimensions: To facilitate effective actuation of the magnet and the magnetic beads, the dimensions of the coil should be smaller than the magnet's diameter but larger than its radius. In some forms, the coil size can be around ⅔ of the magnet's diameter.

Core Material: In PCB (Printed Circuit Board) technology, coils are generally planar and usually do not include a magnetic core due to manufacturing constraints.

Coi Fill Factor: Increasing the coil fill factor helps improve the coil's driving capability, which can be about 50%. To maximize the fill factor, the spacing between the turns should be kept within manufacturable limits, meaning it should be as small as possible (such as about 0.1mm) to increase the proportion of conductive material. Additionally, maximizing the number of coil turns within a limited space can also contribute to a higher fill factor and stronger magnetic field.

Coil Inner Diameter: The inner diameter of the coil can be designed to accommodate a blind via of the minimum size necessary, which is used to connect coils between adjacent layers in series. This design consideration allows that the coil structure remains compact while allowing for effective electrical connections between layers.

The electromagnetic coil array contains 150 or more coils, such as from about 150 to about 300 active coil elements. The number of coil elements required in the electromagnetic coil array depends on the specific application and the range of motion of the magnet. To facilitate continuous and effective magnetic actuation, the array should include enough coil elements to fully cover the magnet's path of movement, allowing for uninterrupted magnetic interaction throughout the operation. Incorporating additional coils offers the benefit of downward compatibility. That is, a coil array with a larger number of elements can be selectively configured or operated to match systems that require fewer coils, providing improved flexibility and adaptability across various biochip designs and use cases.

FIGS. 18D and 18E are a top view and a bottom view respectively, of an exemplary EM chip 120 showing the arrangement of the electromagnetic coils 122 and circuits 124 on the solid substrate 126. The electromagnetic coils 122 are structurally integrated with the solid substrate 126, such as being mounted on or embedded within its surface. The coils 122 are arranged in a defined array or matrix configuration and are spatially aligned with the chambers of the overlying biochip, such as the sample loading chamber, wash chambers, and detection chamber, to facilitate targeted actuation of magnetic beads through the biochip. Each coil 122 can be formed from conductive wire, such as copper, wound around a core, which optionally includes a magnetic material (e.g., ferrite) to improve magnetic field strength. In some forms, each coil element includes a four-turn coil (e.g., 2 mm×2 mm). As shown in FIG. 18E, the bottom side of the EMchip can include passive components 121, such as resistors and capacitors, for power regulation and signal filtering.

### 2. Magnetic Motor

The EM chip includes at least one magnetic motor, configured as a movable magnetic element, such as a permanent magnet or magnetic disc. The size and shape of the magnetic motor generally corresponds with the layout of the coil array, facilitating precise actuation along the surface of the chip via localized magnetic fields generated by the electromagnetic coils.

The magnetic motor is positioned above the array of electromagnetic coils on the surface of the EM chip. In some forms, the magnetic motor can be housed or suspended within an interface region, separating it from direct contact with the coil layer, while maintaining sufficient proximity to respond to magnetic field gradients produced by the electromagnetic coils. As each coil is activated independently via the programmable control circuitry, the magnetic motor is guided across the electromagnetic chip through a series of controlled magnetic pulses, facilitating precise navigation beneath the biochip. The spatial alignment between the magnetic motor and the overlying chambers of the biochip (such as the sample loading, binding, and detection chambers), allows the magnetic motor to transmit mechanical forces to magnetic beads or droplets above, thereby facilitating targeted actuation within the microfluidic device.

For example, as illustrated in FIG. 18F, the EM chip 120 includes at least one magnetic motor 128, configured as a movable magnetic element, such as a permanent magnet or magnetic disc. The size and shape of the magnetic motor 128 are selected to correspond with the layout of the electromagnetic coils 122, to facilitate precise actuation along the surface of the chip through localized magnetic fields generated by the coils. The magnetic motor 128 is positioned above the array of electromagnetic coils 122 on the surface of the EM chip 120. In some embodiments, the magnetic motor 128 is suspended or housed within an interface region, separating it from direct contact with the coil layer while maintaining sufficient proximity to the electromagnetic field gradients produced by the coils. As each coil in the array is independently activated via the programmable control circuitry 124, the magnetic motor 128 is guided across the EM chip in a controlled sequence of magnetic pulses.

### 3. Integrated Circuits

The EM chip includes a plurality programmable switch integrated circuits (ICs) mounted on a lower layer (e.g., between the second layer and the third layer) of the multilayer printed circuit board (PCB). The plurality of ICs can be arranged to provide electrical connectivity and to allow for compact integration within the chip architecture. Each IC can include one or more output ports. Each port can be electrically connected to a corresponding electromagnetic coil element located on one or more upper layers (e.g., the second layer) of the PCB to facilitate activation of specific coils. Optionally, the ICs are electrically connected to the coil elements and other components through conductive traces and vertical interconnects (vias) within the PCB structure. In some forms, for example, the chip can include 20 or more ICs, such as 21 programmable switch ICs (e.g., MAX 14662, Maxim Integrated), arranged in a configuration that supports serial communication protocols, such as Serial Peripheral Interface (SPI).

### 4. Exemplary Electromagnetic Chip

An exemplary electromagnetic chip, the μ-Bead EMchip, is illustrated in FIGS. 18D and 18E. The μ-Bead EMchip is configured to regulate the activation of electromagnetic coils for electromagnetic actuation of the magnetic motor. The μ-Bead EMchip contains a solid substrate containing an array of electromagnetic coils, twenty-one switch integrated circuits electrically connected to the electromagnetic coil array, a magnetic motor, and a microcontroller. The solid substrate for the μ-Bead EMchip is formed of a four-layer printed circuit board (PCB) structure. The PCB substrate includes four conductive and insulating layers. The first PCB layer can be configured to house the magnetic motor, providing structural support and alignment for its movement across the μ-Bead EMchip chip. The second PCB layer can be configured to embed the electromagnetic coil elements, allowing for the generation of localized magnetic fields for actuation of the magnetic motor. The PCB substrate includes a third and fourth layer for mounting integrated circuits (ICs) and managing signal routing and power distribution.

The μ-Bead EM chip includes a magnetic motor, configured as a movable magnetic element, such as a permanent magnet or magnetic disc. The magnetic motor is configured to interface with the μ-Exo biochip positioned above the μ-Bead EM chip. The size and shape of the magnetic motor corresponds with the layout of the coil array on the EM chip, facilitating precise actuation across the surface of the chip through localized magnetic fields generated by electromagnetic coils. The magnetic motor of the μ-Bead EM chip is positioned above the coil array and is housed or suspended within an interface region that separates it from direct contact with the coil layer, while maintaining close proximity to the coils to effectively respond to generated magnetic field gradients. As each coil in the array is independently activated via programmable control circuitry, the magnetic motor can be guided in a stepwise manner across the chip using a series of controlled magnetic pulses.

The motion of the magnetic motor in the μ-Bead EMchip is spatially aligned with the layout of the sample loading chamber, binding chamber, and detection chamber of the μ-Exo biochip. This alignment allows the magnetic motor to transmit mechanical forces through the biochip to manipulate magnetic bead clusters or droplets, facilitating targeted actuation and controlled transport of assay components throughout the microfluidic system.

The μ-Bead EMchip includes an array of electromagnetic coil elements, each configured to generate localized magnetic fields for precise actuation of a magnetic motor. For example, the EM chip includes 165 coil elements each formed as a multi-turn (e.g., four-turn) coil with dimensions of approximately 2 mm ×2 mm, and arranged with narrow spacing (e.g., a 0.1 mm gap) between adjacent coils. The coils may be distributed across multiple upper PCB layers. The array of electromagnetic coils in the μ-Bead EMchip is arranged such that they align with the sample loading chamber, wash chambers, binding chamber, and the detection chamber of the μ-Exo biochip.

Each coil element can be individually manipulated and powered by a direct current (DC) source, for instance, at 0.4 A, to generate sufficient magnetic force to actuate the movable magnetic motor. Control of the coil elements can be achieved through a set of programmable switch integrated circuits (ICs) mounted on a lower PCB layer, with each output port electrically connected to a corresponding coil element. In one example, the EM chip includes 21 switch ICs (e.g., MAX14662, Maxim Integrated) configured in a daisy-chain arrangement using a serial peripheral interface (SPI) protocol.

The ICs can interface with a microcontroller, such as an Arduino Nano, which can be programmed with a path-finding algorithm. This algorithm processes commands received from an external user interface, determines the required coil activation sequence, and transmits corresponding SPI commands to the switch ICs for automated, addressable magnetic actuation.

### C. Optical Sensing Module

The microfluidic device can further include an optical sensing module to measure light absorbance through the signal readout droplet in the detection chamber, thereby allowing quantitative analysis of assay results. The optical sensing module can be integrated into the lower portion of the device, directly beneath the lowest layer of the EM chip, and in vertical alignment with the detection chamber. In some forms, the optical sensing module can be mounted on or embedded within a case enclosing the microfluidic device (see FIG. 1C).

The optical sensing module typically includes a light-emitting component, such as a blue LED with a peak emission wavelength of approximately 450 nm, and a light-detecting component, such as an ambient light sensor with a peak absorbance at approximately 560 nm. The optical sensing module can also include an optical sensor, for or detecting light transmitted through or emitted from a droplet within the detection chamber, such as absorbance, fluorescence, or colorimetric changes that indicate the presence or concentration of a target analyte.

For example, the optical sensing module can include a blue light-emitting diode (LED) (e.g., L450R-04, USHIO), and an optical sensor (e.g., BH1721, ROHM Semiconductor, 3.3 V) for capturing transmitted or absorbed light. The optical sensor can interface with a microcontroller, such as an Arduino Nano, via an Inter-Integrated Circuit (I²C) communication protocol for data transmission and control. In some forms, a 2 mm×2 mm board cutout can be centrally aligned with the detection region of the EMchip (such as an μ-Bead EMchip), allowing the optical path to pass through the droplet without obstruction. During operation, light from the LED travels through the droplet positioned in the detection chamber and reaches the optical sensor through the cutout, facilitating measurement of optical properties such as absorbance or colorimetric changes (see FIG. 18A).

An exemplary optical sensor 130 is illustrated in FIG. 18G. The optical sensor 130 can include a substrate 131 which serves as the structural foundation for one or more integrated components. An optical sensor chip 132 can be mounted on the surface of the substrate 131, and functions as the detection element for capturing and processing light signals. One or more electrically conductive traces 139 can extend from the optical sensor chip 132, forming a circuit that facilitates signal transmission. These electrically conductive traces 139 connect the optical sensor chip 132 to a connection interface 138 containing multiple terminals for power and data communication. For example, the connection interface 138 can include a ground terminal (GND) 133 for electrical stability, a reset pin (RST) 134 for initializing the sensor, a serial clock line (SCL) 135, a serial data line (SDA) 136 for communication via an I²C protocol, and a power supply terminal (VCC) 137 to provide operating voltage.

### D. Optional Components

The microfluidic system can include one or more optional components such as a user display, a case, and/or reagents and/or buffers. In some forms, the one or more reagents and/or buffers can be provided as separate components in the package of the microfluidic system.

### 1. Reagents and Buffers

The microfluidic systems can include one or more reagents and/or buffers. Reagents used for detection and analysis of the analyte can be prepositioned in the detection chamber of the biochip (e.g., capture agents immobilized on the analysis area substrate). For example, reagents can be introduced into the detection chamber at the time of performing the assay or pre-loaded into the detection chamber prior to performing the assay. The reagents suitable for use in the detection chamber can react with the reactive label of the binding agent used in the binding chamber, the reaction between which can generate a readable signal, such as an optional signal. The buffers used in the microfluidic systems are generally suitable for washing the immunomagnetic beads to remove non-specifically bound non-target molecules and/or binding agents. Suitable buffers for use in the washing chambers of the biochip include, but are not limited to phosphate buffered saline.

In some forms, the microfluidic systems include one or more imaging agents. Imaging agents are compounds or molecules that include or produce a detectable label. A detectable label refers to a compound or molecule capable of emitting or generating a measurable signal. In some forms, the imaging agent may specifically bind to an assay molecule, or to a complex or product formed during the assay. This interaction facilitates the label to become localized within a specific region of the detection chamber of the biochip—such as where the assay molecule binds or where a reaction product accumulates—allowing the emitted signal to be spatially correlated with a particular analytical event.

Examples of suitable imaging agents include but are not limited to fluorescent labels such as fluorescein (FITC), 5,6-carboxymethyl fluorescein, Texas Red, nitrobenz-2-oxa-1,3-diazol-4-yl (NBD), coumarin, dansyl chloride, rhodamine, 4′,6-diamidino-2-phenylindole (DAPI), and cyanine dyes such as Cy3, Cy3.5, Cy5, Cy5.5, and Cy7. Fluorescent labels for general use include but are not limited to 5-carboxyfluorescein-N-hydroxysuccinimide ester and 5,6-tetramethyl rhodamine, while FITC and cyanine dyes are particularly suitable for combinatorial multicolor coding. Their respective excitation and emission maxima are: FITC (490 nm; 520 nm), Cy3 (554 nm; 568 nm), Cy 3.5 (581 nm; 588 nm), Cy5 (652 nm; 672 nm), Cy5.5 (682 nm; 703 nm), and Cy7 (755 nm; 778 nm). In some forms, the optical module is configured to measure light absorbance within the biochip. However, if the microfluidic device is to be adapted for the detection of fluorescent signals, simple optical filters or lenses can be integrated into the module to selectively capture emitted fluorescence while blocking excitation light.

### 2. Case

The microfluidic system is optionally enclosed in a case, e.g., a portable case. For example, the microfluidic system can be provided as a point-of-care device. The case can be formed from any material which provides protection against mechanical shock, moisture, and environmental contaminants during handling and operation outside of laboratory settings. For example, the case can be formed from lightweight, impact-resistant materials, such as polycarbonate, ABS plastic, or aluminum alloys. In one exemplary form, the case is formed of resin such as Tough 2000 Resin.

In some forms, the length of the case ranges from about 100 mm to about 150 mm, such as about 110 mm, about 120 mm, or about 130 mm. In some forms, the width of the case ranges from about 60 mm to about 80 mm, such as about 65 mm, about 70 mm, or about 75 mm. In some forms, the height of the case ranges from about 30 mm to about 50 mm, such as about 35 mm, about 40 mm, or about 50 mm. In one exemplary form, the case has a length, width, and height of about 127 mm, 71 mm, and 36 mm respectively.

The case can include a base section configured to securely hold the internal components of the system, such as the biochip, electromagnetic chip, optical sensor module, and control electronics, with integrated mounting features or internal compartments to maintain alignment. A top cover may include access points for sample loading and transparent windows or cutouts aligned with the detection chamber for visual inspection or optical readout. The cover may be attached via a hinge or latch mechanism for quick opening and closing in field applications.

For usability in mobile settings, the case can incorporate ergonomic design features, such as a carrying handle, low-profile form factor, and non-slip base, ensuring stable placement and easy handling. Ventilation openings may be included to dissipate heat generated by onboard components during operation. The portable case enables reliable, user-friendly deployment of the microfluidic device in remote, clinical, or resource-limited environments.

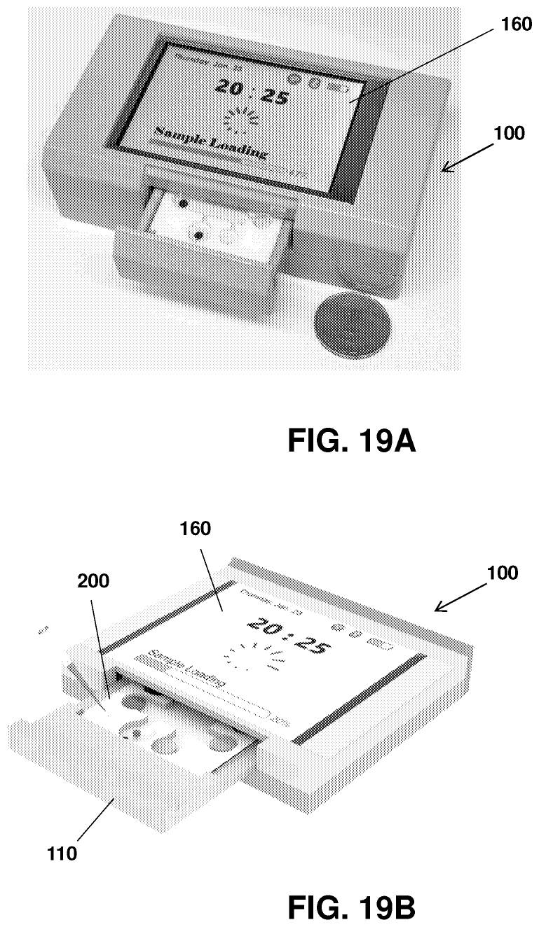

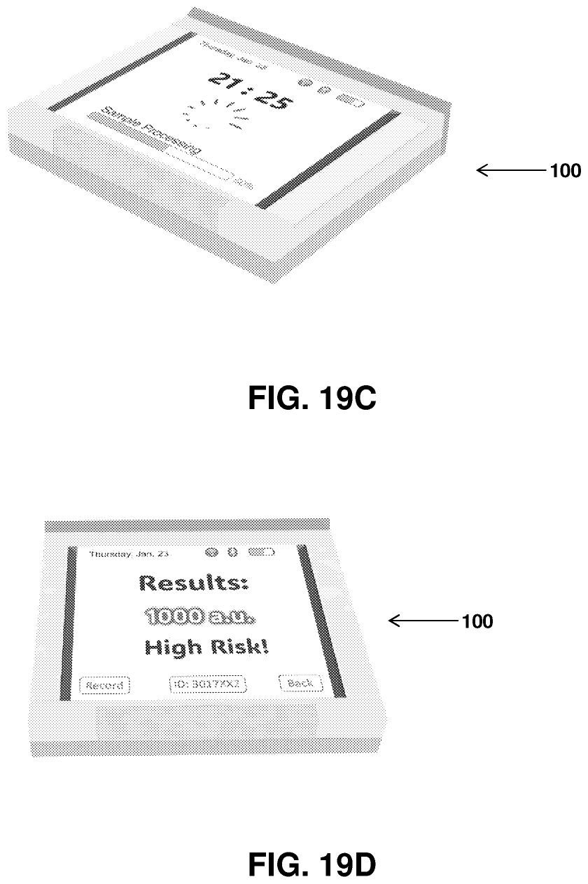

As shown in FIGS. 19A-19D, an exemplary case 100 can include a user interface 160 integrated into the upper surface of the case 100. The user interface 160 can be in the form of a touchscreen interface, configured to present real-time system status, assay progress, and user prompts (FIGS. 19A-19D). In some forms, the screen displays the current date and time, Wi-Fi connectivity, battery status, and a progress indicator corresponding to a “Sample Loading” step (see FIGS. 19A and 19B). The user interface 160 allows users to interact with the device via a graphical user interface (GUI) and can also support touch-based input for navigation through assay workflows.

The case 100 can include a compartment 110 configured to receive and secure the biochip 200 (see FIG. 19B). The compartment 110 can be housed in a drawer-style or sliding compartment that allows the user to insert the biochip 200 manually. In the closed position, the compartment 110 can be aligned with the underlying electromagnetic chip and optical sensing module, such that that the relevant chambers within the biochip, such as the sample loading chamber, binding chamber, and detection chamber, are properly positioned above the corresponding functional components within the device. The compartment 110 can also include features such as guide rails, alignment pins, or recessed seating structures for stable placement and electrical or optical connectivity if needed.

### 3. User Display/interface

The microfluidic system optionally includes a user interface/user display that can be manipulated by an operator. Optionally, the microfluidic system includes a user interface for operating and controlling one or more device functions. In some forms, the upper surface of the device housing can incorporate a user interface panel configured to provide access to system controls, such as initiating or pausing fluidic operations, starting assays, or displaying real-time status indicators. The user interface can be connected to internal components of the device—such as the electromagnetic chip, optical sensor, or fluid control modules—via wired or wireless connections, including Bluetooth, Wi-Fi, or serial communication protocols.

The user interface may include one or more interactive controls, such as push buttons, touch-sensitive buttons, LED backlit buttons, rotary knobs, or capacitive touch sliders, allowing users to control various operational parameters without opening the device. For example, a push button may be provided to start or stop an assay cycle, while touch-sensitive areas can be designated for specific commands like initiating sample loading or triggering the readout process. In some forms, LED backlit buttons are used to indicate different operational states—such as assay in progress, completed, or error—through changes in color or brightness. A rotary knob can optionally be included to allow users to navigate menu options or adjust settings such as assay duration or sensor calibration parameters. Alternatively, a capacitive slider may be integrated, enabling users to adjust values (e.g., motor speed or sensitivity thresholds) by sliding a finger across a designated interface zone.

In some forms, the user interface is provided through a touchscreen display, which can function as an interactive control panel and a visual output module, or both. The touchscreen can allow users to initiate assay runs via on-screen buttons and save or record test results upon completion of the assay. The interface can also guide users through various stages of the assay using interactive prompts, status messages, and progress indicators. Multiple interface screens can be displayed throughout the workflow, including sample loading, processing, and result visualization. For example, the results from the assay can be immediately provided on the user display as shown in FIG. 19D.

In other forms, the user interface may be omitted from the system, and the microfluidic system can instead be remotely controlled via an external interface, such as a wireless handheld remote or a software application running on a smartphone, tablet, or computer. This remote interface can provide functions such as power on/off, protocol selection, progress monitoring, and data export, enabling users to interact with the device conveniently and without direct physical contact.

### II. Methods of Using the Memos

Also provided are methods of detecting one or more analytes in a biological sample. Generally, detection method using the microfluidic system is highly automated and the user simply loads the biological sample into the sample loading chamber of the biochip and then starts the system by generating an electromagnetic field. Optionally, before sample loading, the user loads the immunomagnetic beads, one or more wash buffers, one or more binding agents, one or more reactants, and/or a stop buffer into the respective chambers of the biochip. Optionally, the user prepares the buffers and/or reactants for loading into the chambers of the biochip.

The electromagnetic filed generated by the EMchip directs the flow of the magnetic beads through the different chambers on the biochip to conduct the assay. For example, after pressing a button incorporated in or externally connected to the microfluidic system, the generated electromagnetic field directs mixing of the immunomagnetic exosome beads loaded in the sample loading sample with the sample to allow binding of one or more target analytes to the first binding agent on the surface of the magnetic beads to form bead-analyte complexes. The bead-analyte complexes are directed to an adjacent wash chamber and mixed with the wash buffer contained therein to remove non-specifically bound molecules. The washed bead-analyte complexes are subsequently directed to the binding chamber containing a second binding agent labeled with a reactive label to allow binding of the second binding agent to the one or more target analytes to form bead-analyte label complexes. The bead-analyte complexes label complexes are directed to one or more adjacent wash chambers and mixed with the wash buffers contained therein to remove non-specifically bound binding agents. The washed bead-analyte label complexes are then directed to flow into the detection chamber containing one or more reactants for signal generation. The reactants in the detection chamber can react with the reactive labels on the second binding agent and generate a readable signal, such as an optical signal, which can be detected and quantified by an optical module included in the microfluidic system.

The biological sample can be any suitable biological sample such as tissue, blood, plasma, serum, saliva, urine, cerebrospinal fluid (CSF), breast milk, and ascitic fluid. Generally, the biological sample is obtained from a mammal such as a human, a rodent, a domestic animal, a pet, or a wild animal. In some forms, the biological sample is a plasma sample or a serum sample from human.

Typically, the microfluidic system allows detection with the use of a very small amount of biological sample, such as less than 200 μL, such as less than 100 μL or less than 20 μL or less than 10 μL. In some forms, the biological sample is fluid sample ranging from 1 μL to 200 μL. For example, In some forms, the biological sample is fluid sample ranging from about 5 μL to about 150 μL, about 5 μL to about 100 μL, about 5 μL to about 80 μL, about 5 μL to about 60 μL, about 5 μL to about 40 μL, about 5 μL to about 20 μL, or 5 μL to about 10 μL.

The microfluidic devices, automated systems, and methods of use thereof can be further understood by the following numbered paragraphs.
Paragraph 1. A microfluidic device for automated analyte detection comprising:
   (a) a biochip;
   (b) an electromagnetic chip comprising a programmable navigation matrix;
   wherein the biochip and the electromagnetic chip are oriented parallel to each other, and
   wherein the electromagnetic chip is directly below the biochip.
Paragraph 2. The microfluidic device of paragraph 1, wherein the biochip comprises:
   (i) a sample loading chamber;
   (ii) one or more wash chambers;
   (iii) a binding chamber; and
   (iv) a Detection Chamber;
   wherein the sample loading chamber, one or more wash chambers, the binding chamber, and the detection chamber are in fluid communication with each other.
Paragraph 3. The microfluidic device of paragraph 1 or 2, further comprising a plurality of immunomagnetic exosome beads; optionally wherein the plurality of immunomagnetic exosome beads is provided as a separate component or is pre-loaded in the sample loading chamber.
Paragraph 4. The microfluidic device of any one of paragraphs 1-3, wherein the biochip comprises two or more wash chambers,
   wherein a first wash chamber is in direct fluid communication with the sample loading chamber and the binding chamber, and
   wherein a second wash chamber is in direct fluid communication with the detection chamber and the binding chamber.
Paragraph 5. The microfluidic device of any one of paragraphs 2-4, wherein the biochip further comprises a stop buffer chamber, and wherein the detection chamber is in direct fluid communication with the stop buffer chamber.
Paragraph 6. The microfluidic device of any one of paragraphs 1-5, wherein the electromagnetic chip comprises a programmable navigation matrix comprising:
   (1) a plurality of electromagnetic coils,
   (2) one or more switch integrated circuits electrically connected to the plurality of electromagnetic coils,
   (3) one or more magnetic motors, and
   (4) a microcontroller configured to regulate the activation of electromagnetic coils for electromagnetic actuation of the magnetic motor.
Paragraph 7. The microfluidic device of paragraph 6, wherein the plurality of electromagnetic coils are arranged such that they align with the sample loading chamber, the one or more wash chambers, the binding chamber, and the detection chamber.
Paragraph 8. The microfluidic device of paragraph 6 or 7, the plurality of electromagnetic coils are arranged such that they align with the stop buffer chamber.
Paragraph 9. The microfluidic device of any one of paragraphs 1-8, further comprising a material exchange area,
   wherein the material exchange area is in fluid connection with the sample loading chamber, the one or more wash chambers, the binding chamber, and the detection chamber, and
   optionally, wherein the material exchange region is centrally positioned on the biochip.
Paragraph 10. The microfluidic device of any one of paragraphs 2-9, wherein each of the sample loading chamber, the one or more wash chambers; the binding chamber, and the detection chamber comprise a mixing area and a flow region.
Paragraph 11. The microfluidic device of any one of paragraphs 1-10, further comprising:
   (c) an optical sensing module; and
   (d) optionally an operation module.
Paragraph 12 The microfluidic device of paragraph 11, wherein the optical sensing module comprises:
   (i) a light source aligned with the detection chamber to emit light through the signal readout droplet;
   (ii) a photodetector configured to receive transmitted or absorbed light from a droplet in the detection chamber and generate an output signal corresponding to the transmitted or absorbed light levels; and
   (iii) optionally, a supporting structure to secure the optical sensing module in fixed alignment with the detection chamber.
Paragraph 13. The microfluidic device of paragraph 11 or 12, wherein the light source of the optical sensing module is aligned below the detection chamber.
Paragraph 14. The microfluidic device of any one of paragraphs 1-13, wherein the biochip comprises
   (a) one or more barrier layers, comprising a top layer and a bottom layer;
   (b) one or more chamber layers; and
   (c) optionally one or more adhesive layers,
   wherein when the biochip is assembled the one or more chamber layers form one or more chambers and one or more flow paths,
   and the one or more chamber layers and one or more adhesive layers are positioned between the top layer and the bottom layer.
Paragraph 15. The microfluidic device of paragraph 14, wherein the one or more barrier layers are formed of one or more polymers selected from the group consisting of polyethylene terephthalate, polycarbonate, cyclic olefin copolymer, cyclic olefin polymer, poly(methyl methacrylate), polyethylene naphthalate (PEN), clarified polypropylene, fluoropolymers such as Cytop, cellulose acetate Films and thermoplastic polyurethane.
Paragraph 16. The microfluidic device of paragraph 14 or 15, wherein the one or more chamber layers are formed from one or more polymers selected from Polytetrafluoroethylene, DMS, cyclic olefin copolymer, cyclic olefin polymer, poly(methyl methacrylate), polycarbonate, clarified polypropylene, Fluorinated Ethylene Propylene, Polyethylene Naphthalate (PEN), and TPU.
Paragraph 17. A method for detecting an analyte in a biological sample using the microfluidic device of any one of paragraphs 1-16.
Paragraph 18. A method for detecting an analyte in a biological sample comprising the steps of:
   (a) loading the biological sample into the sample loading chamber of the biochip of the microfluidic device of any one of paragraphs 1-16; and
   (b) initiating the generation of electromagnetic field.
Paragraph 19. The method of paragraph 17 or 18, wherein the plurality of immunomagnetic exosome beads is functionalized with a binding agent that specifically bind one or more exosomal markers.
Paragraph 20. The method of paragraph 19, wherein the exosomal marker is selected from the group consisting of CD63, CD9, CD81, CD82, CD151, Anti-HSP70, anti-HSP90, Annexin V Tim4 protein, or a combination thereof.
Paragraph 21. The method of paragraph 19 or 20, wherein the binding agent is selected from antibodies, aptamers, nanobodies, peptides, and ligands.
Paragraph 22. The method of any one of paragraphs 17-21, wherein the biological sample is selected from the group consisting of tissue fluid, blood, plasma, serum, saliva, urine, cerebrospinal fluid (CSF), breast milk, and ascitic fluid.
Paragraph 23. The method of any one of paragraphs 17-22, wherein the biological sample is a plasma sample or a serum sample.
Paragraph 24. The method of any one of paragraphs 17-23, wherein the biological sample is fluid sample ranging from 1 μL to 500 μL.
Paragraph 25. The method of any one of paragraphs 17-24, wherein the biological sample is obtained from a mammal.
Paragraph 26. The method of any one of paragraphs 17-25, wherein the biological sample is obtained from a human, a rodent, a domestic animal, a pet, or a wild animal.
Paragraph 27. The method of any one of paragraphs 17-26, wherein the immunomagnetic exosome beads are pre-loaded into the biochip prior to sample loading or wherein the immunomagnetic exosome beads are provided as a separate component.

More detailed descriptions of the methods for using the microfluidic devices are provided in the non-limiting Examples below.

### EXAMPLES

### Materials and Methods

### Materials

Plasma sample from healthy individuals, Alzheimer's disease patients, and patients with mild cognitive impairment were obtained from Innovative Research, Inc. (Novi, MI, USA), BioIVT LLC. (Westbury, NY, USA) and Molecular Depot LLC (San Diego, USA) to minimize the bias in the plasma sample treatment process. Mild cognitive impairment represents an early stage of memory loss, characterized by less severe symptoms compared to Alzheimer's disease, which is confirmed through Mini-Mental State Examination (MMSE) screening. The confirmation of AD is based on clinical diagnosis. All human plasma samples were stored at −20 °C. Human primary anti-CD63 antibody (MA5-30187), APC goat anti-Rabbit IgG (H+L) Secondary Antibody (A-10931), and 10 nm AuNPs conjugated goat anti-Rabbit IgG (H+L) secondary antibody (A-31566), were obtained from ThermoFisher Scientific. Mouse primary anti-CD63 antibody (ab281516), mouse anti-beta Amyloid 1-42 antibody (ab280993), and human anti-beta Amyloid 1-42 antibody (ab240233) were obtained from Abcam limited. Total Exosome Isolation Reagent (from cell culture media) (4478359) was obtained from ThermoFisher Scientific. Exosome FITC staining (EXOFLOW800A-1) and the FluoroCet Exosome Quantitation Kit (FCET96A-1) were obtained from System Biosciences.

### Cell Culture

Human neuroblastoma cell line SH-SY5Y was obtained from the American Type Culture Collection (ATCC). SH-SY5Y cells, at passages 1-15, were cultured in a medium consisting of Minimum Essential Medium (MEM, Gibco) and Ham's F-12 Nutrient Mix with GlutaMAX™ Supplement (F-12, Gibco) in a 1:1 ratio. The culture medium was supplemented with 10% exosome-depleted fetal bovine serum (EXO-FBS-250A-1, System Biosciences) and 1 % penicillin-streptomycin (15070063, Gibco). The cells were maintained at 37 °C in a humidified atmosphere containing 5% CO₂.

### Exosome Isolation

SH-SY5Y cells at passages 1-15 were cultured in exosome depleted medium with 10% exosome-depleted FBS (EXO-FBS-250A-1, System Biosciences) for 48 h. The cell culture media was harvested and filtered through a 0.2-μm membrane filter. The filtered media was then centrifuged at 2000×g for 30 mins to remove cell debris. The supernatant was transferred to a new tube, mixed with total exosome isolation reagent (4478359, Invitrogen) according to the manufacture's protocol, and incubated at 4 °C overnight. Following incubation, the sample was centrifuged at 10000×g for 1 h at 4 °C. The supernatant was discarded, and the exosomes were collected in the pellet at the bottom of the tube. PBS was used to resuspend the exosome pellet for subsequent experiments.

### Preparation of iME Beads-based Exosome Identification Assay

The anti-CD63 primary antibody was first conjugated with biotin using a commercial biotinylation kit (ab201796, Abcam) following the protocol. After conjugation, biotin-conjugated CD63 primary antibody (0.07 mg/ml) was incubated with streptavidin-coated magnetic beads (4 mg/ml, 11206D, Invitrogen) in 100 μl PBS for 2 hours at room temperature under constant rotation and protected from light. Then, the beads were washed three times in PBS containing 0.4 % Block Ace (Bio-Rad), and blocked with PBST buffer, containing 4% Block Ace for 1 hour at room temperature. After blocking, the coated beads were concentrated to 10 mg/ml in buffer A SignalPlus Antibody Enhancer (GeneTex) to enhance the binding affinity. The iME beads were then ready to use for exosome capture. To generate a signal, Horseradish peroxidase (HRP) was conjugated to the anti-Aβ42 antibody using a commercial HRP antibody conjugation kit (ab102890, Abcam) according to the protocol. The HRP conjugated anti-Aβ42 antibody was diluted 1:1000 in buffer B SignalPlus Antibody Enhancer (GeneTex) before use. Wash buffer was prepared using PBST containing 0.4% Block Ace. TMB buffer (34028) and stop buffer (SS03) were purchased from ThermoFisher Scientific. For each test, the sample was loaded and mixed with 8 μl of iME beads on the chip for 1 hour. After that, the beads were transferred to a 50 μl wash buffer droplet and washed for 5 minutes. The beads were then mixed and incubated with 50 μl droplet of HRP-conjugated anti-Aβ42 antibody for 1 h, followed by three washes. Next, the beads were extracted to react with a 50 μl TMB droplet for 15 minutes. Finally, a 50 μL stop buffer droplet was added to terminate the reaction before readout. Non-coated polystyrene magnetic microbeads (49664, Sigma-Aldrich) were used to adjust the bead loading mass, facilitating the operation of beads and droplets.

### Fabrication of the Microfluidic Exosome Detection Biological Chip (μ-Exo Biochip)

The μ-Exo Biochip was fabricated by laser engraving different patterns on two polytetrafluoroethylene (PTFE) substrates, which were then sandwiched between two transparent polyethylene terephthalate (PET) film sheets. This configuration encapsulated mineral oil (5904, Sigma-Aldrich) and assay reagents within a sealed environment. The substates were assembled by using double-sided tape to construct chamber heights to of approximately 3 mm.

### Design of the Microbead Operation Electromagnetic Chip (μ-Bead EMchip)

The μ-Bead EMchip was designed based on a four-layer PCB⁵⁷ and consists of 165 active coil elements. Each element features a four-turn coil (2 mm×2 mm) that is stacked on the top three layers of the PCB, with a 0.1-mm gap separating it from adjacent elements. Each coil element can be powdered independently by a direct current (DC) of 0.4 A, generating a localized magnetic force to attract the magnetic motor. A total of 21 programmable switch ICs (MAX14662, Maxim Integrated), were mounted on the bottom layer of the PCB, with each output port connected to individual coil element to selectively activate the coil elements. The switch ICs were connected in a serial peripheral interface Daisy-Chain configuration to Arduino Nano, which communicates with a computer through serial communication. A simple path-finding algorithm was embedded in the Microcontroller. Target destinations or operations sent from the user interface were processed by the path-finding algorithm, then translated into serial peripheral interface commands by the Arduino and subsequently transmitted to the switch ICs for automated addressable activation of the coil elements.

A blue LED with a peak wavelength of 450nm (L450R-04, USHIO) and an ambient light sensor (3.3 V) with a 560 nm peak absorbance (BH1721, ROHM Semiconductor) were used to measure the optical signal. The ambient light sensor was connected to Arduino Nano through Inter-Integrated Circuit protocol (I2C). A 2 mm×2 mm board cutout was centrally positioned within the detection region of the μ-Bead EMchip. The light emitted by the LED passed through the droplet and then reached the ambient light sensor through the cutout. To ensure measurement accuracy, light intensity was sampled and filtered continuously over a duration of 10 seconds.

### Preparation of Synthesized Exosome-amyloid Beta Associations

Lyophilized Aβ42 protein (rPeptide) was resuspended in PBS buffer, containing the exosome-isolated pellet, and rotated at 4 °C for 20 h to facilitate the formation of associations. After incubation, the initial association buffer was diluted with PBS to achieve the desired total Aβ42 concentration and was used immediately. Aliquots of 100 μl were used for on-chip testing.

### Flow Cytometry Analysis

To characterize the immune coating efficiency of magnetic beads, an APC-conjugated secondary antibody was used to label the primary antibody. Briefly, primary antibody coated beads (100 μg/100 μl PBS) were incubated with secondary antibody (10 μg/ml) for 1 h at room temperature under constant rotation and protected from light. Blank non-coated beads were used as controls for the non-specific binding test. Then, the beads were washed three times in PBS containing 0.4 % Block Ace, after which the pellets were resuspended in 1 ml PBS for flow cytometry analysis using a BD FACSAria III Cell Sorter. To assess the exosome capture performance of the immune beads, a commercial exosome purification kit was used. Briefly, the exosome-captured beads (100 μg) were resuspended in 250 μl of exosome staining buffer containing 10 μl FITC exosome stain and placed on ice for 3 h. The tube was flicked every 30 mins to ensure gently mixing. Blank primary antibody-coated beads were used as controls for the non-specific FITC exosome dye binding test. The beads were then washed three times in PBS containing 0.4 % Block Ace, after which the pellets were resuspended in 1 ml PBS for flow cytometry (BD FACSAria III Cell Sorter). Analyses were performed using FlowJo V10 Software, with single beads gated based on their forward and side scattering properties.

### Electron Microscopy

The energy dispersive X-ray spectroscopy images of the AuNPs conjugated secondary antibody labelled iME beads were obtained using a FEG Scanning Electron Microscope (Hitachi S-4800) without Au sputtering on the samples. Exo-Aβ samples were fixed with 4 % paraformaldehyde (I28800, ThermoFisher Scientific), washed three times, and transferred onto a nickel grid. After that Aβ42 proteins were subject to double immune-labelling using Aβ42 primary antibody (ab180956, Abcam) and 10 nm AuNPs conjugated secondary antibody (A-31566, ThermoFisher Scientific). The labelled samples were washed and contrast stained. Dried samples were imaged with a Scanning Transmission Electron Microscope (FEI Tecnai G2 20). Exosomes and Aβ42 aggregates were fixed with 4% paraformaldehyde and transferred onto a copper grid, washed and contrast stained. Dried samples were imaged with a transmission electron microscope (Philips CM100).

### Confocal Microscopy

The fluorescence images of the fluorescent beads were obtained using a confocal and multiphoton microscope (Nikon NEW AX R high speed confocal, Nikon Ti2-E motorized inverted microscope) by depositing the beads onto a 0.17 mm confocal glass slide. The fluorescence images were recorded by collecting light in the 500-600 nm under excitation by a 488 nm laser.

### Animal Sample Analysis

C57BL/6J mice and 5×FAD mice aged between 7 and 9 months were housed in the Centre for Comparative Medicine Research (CCMR) at the University of Hong Kong, which is accredited by the Association for Assessment and Accreditation of Laboratory Animal Care International. The use of animals was approved by the Department of Health, Hong Kong, and the Committee on the Use of Live Animals in Teaching and Research (CULATR) at the University of Hong Kong (#12-127). Mice were housed in a temperature-controlled room between 20 and 22 °C, with humidity of 50±10% under a 12/12 h light/dark cycle. Food and water were provided ad libitum, and all possible efforts were made to minimize the number of animals being used and animal suffering. 0.6 ml of blood was collected via cardiac puncture into Eppendorf tubes precoated with 0.5 M EDTA. Blood was then centrifuged at 1200×g for 15 mins at 4 °C. Plasma was collected carefully and stored at −80 °C until use. 100 μl aliquots of mice plasma were thawed to room temperature and then loaded onto the chip for testing.

### Human Plasma Samples Analysis

Plasma samples from healthy individuals, Alzheimer's disease patients, and mild cognitive impairment patients were thawed to room temperature before use. 100μl aliquots of human plasma were loaded onto the chip for testing.

### Statistical Analysis

All statistical analyses were performed using GraphPad Prism software version 10.0. Animal samples (n=20) from 5×FAD transgenic mice and C57BL/6J mice, along with clinical samples (n=60) were analyzed using unpaired Mann-Whitney nonparametric test. p<0.05 was considered statistically significant for all analyses.

### Results

### MEMOs Assay Development and Characterization

To detect the target biomolecular Exo-Aβ42 with high sensitivity and automated processing, an iME beads-based Exo-Aβ42 identification assay with dual marker sites targeting capability was developed to specifically identify both the exosomal maker and the amyloid beta marker respectively (FIGS. 1A-1J). Magnetic microbeads are selected as the solid substrate to provide the assay with magnetic field-responsive properties, enabling subsequent downstream isolation, extraction and transportation between multiphase interfaces^{44,45}. Meanwhile, the use of magnetic microbeads also improves the availability of immobilization sites for biomolecules because of their extensive surface area^{46,47}. A sequence of assay processing steps was meticulously developed and systematically evaluated to achieve target molecular identification, consisting of iME beads development, exosome capture, and Aβ42 recognition and signal generation.

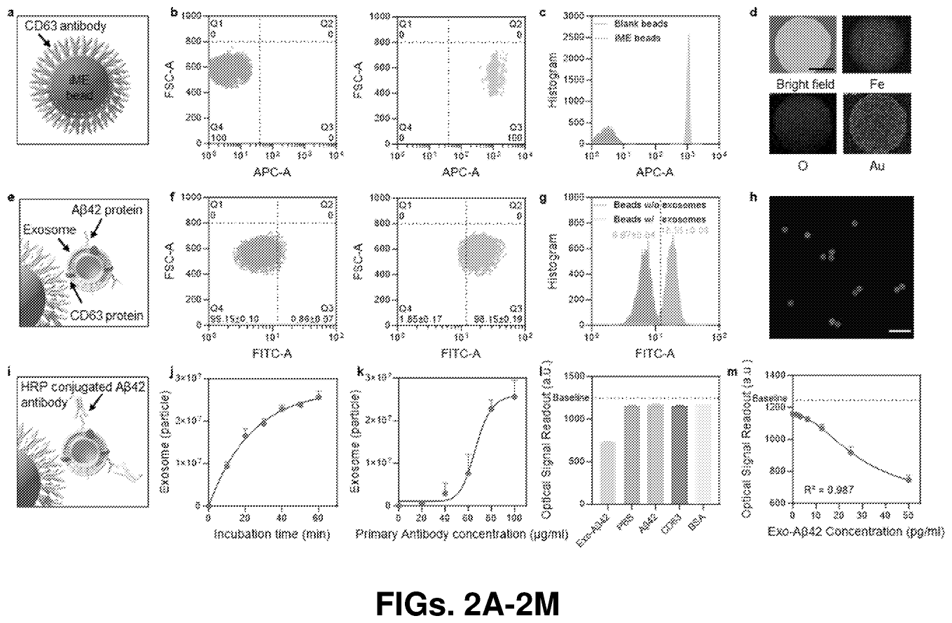

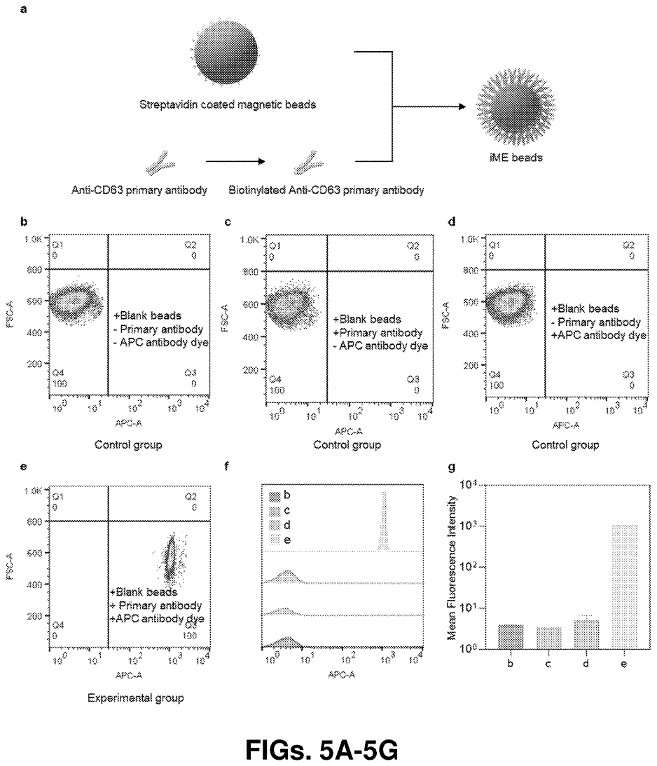

To develop iME beads with high selective isolation capability, typical exosome marker CD63 is targeted (FIG. 2A; FIG. 5A). CD63, a tetraspanin protein that is highly concentrated in the exosomal membrane, is widely utilized in the immunoisolation of exosomes^{37,48,49}. Biotinylated anti-CD63 antibodies are conjugated to the surface of streptavidin-positive magnetic beads via non-covalent interactions for the purpose of exosome identification and enrichment. The immunological functionalization efficiency at the single bead level was characterized by flow cytometry (FIGS. 2B and 2C). Allophycocyanin (APC) fluorescence dye was used to label the CD63 antibody, thereby facilitating the characterization of functionalization efficiency. After gating, the quartile distribution within the dot plot, based on the fluorescent intensity of individual beads, revealed that blank beads are primarily distributed in the fourth quadrant Q4, while iME beads were dispersed in the third quadrant Q3. Compared to blank beads, the APC fluorescence signal of the iME beads shifted significantly, whereas the forward scattering (FSC) signal remained unchanged. The APC signal shift confirms effective antibody immobilization on individual beads, while the unchangeable FSC signal indicated that the immunological coating does not cause any detectable size change of the beads. The histogram of the APC signal for single bead revealed a narrow peak width for the iME beads group, indicating the homogeneity of immunological functionalization. No cross reaction was detected between blank streptavidin beads and APC antibody staining (FIGS. 5A-5G). The functionalization performance is further confirmed by scanning electron microscope (SEM), where an AuNPs-conjugated secondary antibody is used to mark the surface functionalized sites of the iME beads. The results revealed that AuNPs are uniformly localized on the surface of the iME beads, indicating the successful conjugation of the immune coating (data not shown). Energy dispersive X-ray spectroscopy obtained via SEM demonstrated that Au element is uniformly distributed and co-localized with Fe and O elements on the beads surface (FIG. 2D), whereas in the control group, the Au element is not detected (data not shown).

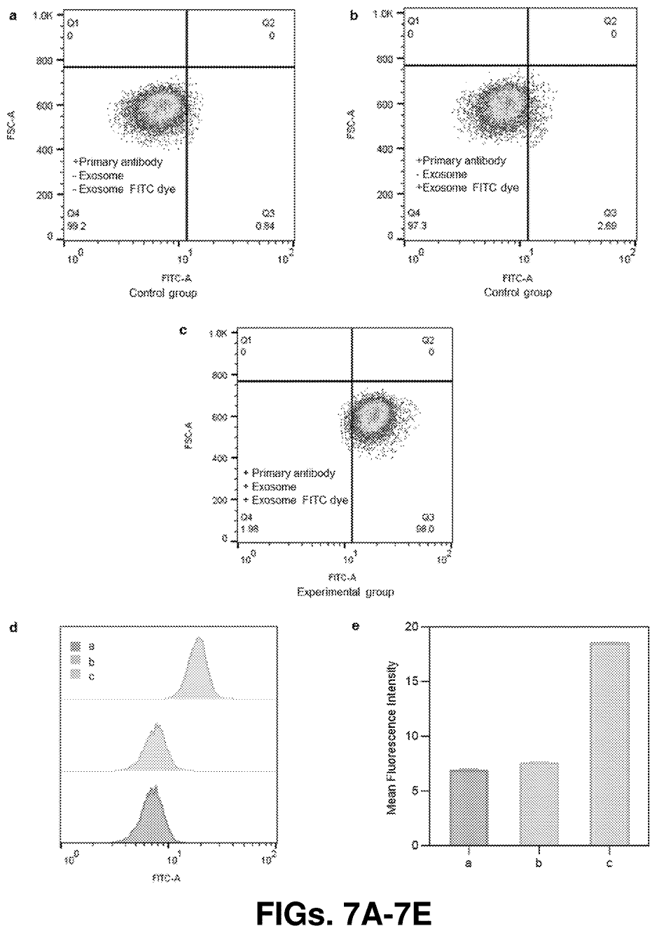

To evaluate the exosome immunocapture performance of iME beads, exosomes isolated from the human neuroblastoma cell line SH-SY5Y (a common cell model used to study molecule events leading to Alzheimer's disease⁵⁰), were employed for in vitro studies (FIG. 2E). The isolated vesicles exhibited a characteristic exosomal cup-shaped morphology under transmission electron microscopy (TEM) (FIG. 6A) and display a uniform distribution of particle sizes, with an average diameter of approximately 150 nm, as measured by nanoparticle tracking analysis (NTA) (FIG. 6B and data not shown). Western blotting confirmed that the exosome-specific protein markers, including the target CD63 protein and the reference TSG101 protein, are prominently expressed in the isolated vesicles (FIG. 6C). The performance of exosome capture was investigated through flow cytometry (FIGS. 2F and 2G and FIGS. 7A-7E) using FITC exosome staining. The quartile distribution revealed that 99.15±0.10% of iME beads before exosome capture were distributed in the Q4 zone, while after incubated with exosomes, 98.15±0.19% of the beads shift to the Q3 zone, indicating the effectiveness of exosome capture. The histogram of the FITC signal for individual beads shows that the mean fluorescence intensity increases from 6.97±0.04 to 18.55±0.06, further suggesting the immune affinity of iME beads for exosome isolation. Confocal microscope images displayed a uniform FITC signal at the surface of the exosome-captured beads (FIG. 2H) and no FITC signal in the control group (data not shown), indicating the uniformity of the capture capability of the iME beads.

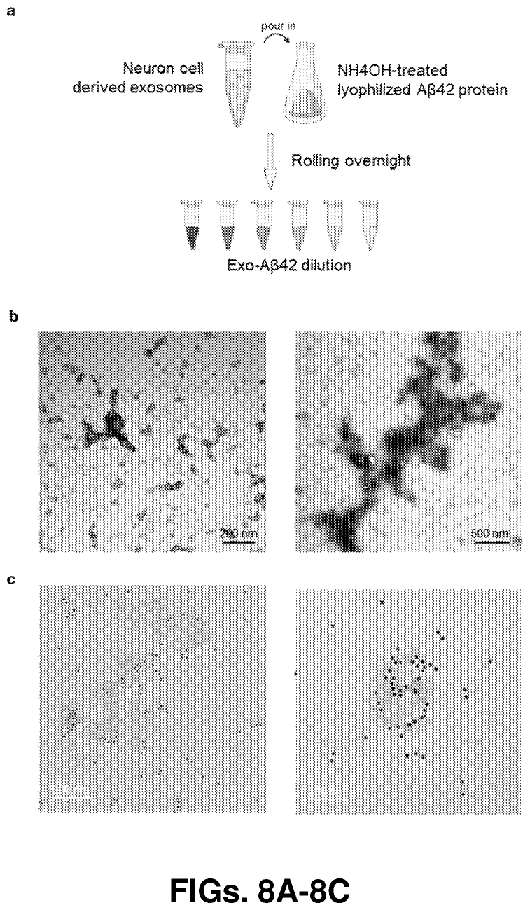

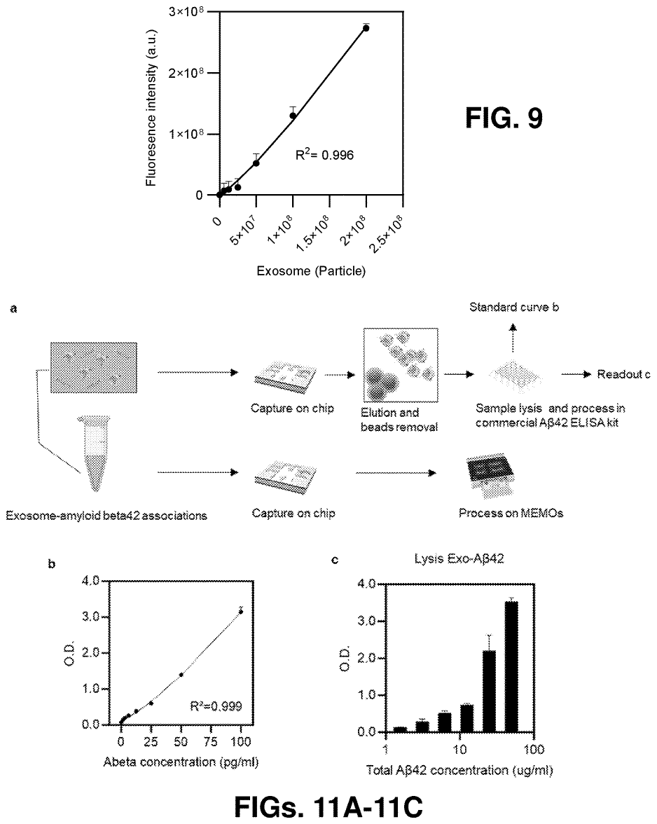

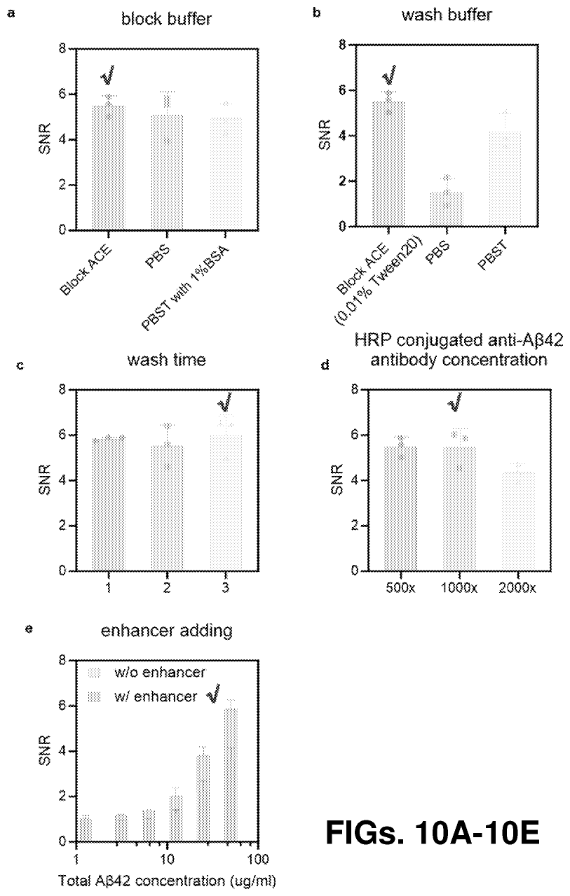

To process Aβ42 recognition and enzymatic signal generation procedures, a horseradish peroxidase (HRP) enzyme-conjugated capture antibody was prepared to target the second marker site, the Aβ42 protein (FIG. 2I). Its substrate 3,3′-diaminobenzidine tetrahydrochloride (TMB) was used to amplify the optical signal. To facilitate in vitro experiments, the exosome-amyloid beta associations (Exo-Aβ42) are prepared by self-assembling Aβ42 aggregates onto the membranes of exosomes (FIG. 8A). The morphology of these associations was confirmed through transmission electron microscopy and scanning transmission electron microscopy (FIGS. 8B and 8C). Notably, both the exosome-amyloid beta associations and free amyloid beta aggregates co-existed in the prepared sample, implying that Exo-Aβ42 represents a subpopulation within the total amyloid beta environment. Using the prepared Exo-Aβ42 biomarkers, the conditions for the exosome capture process were examined, including immune functionalization durations (FIG. 2J) and concentrations (FIG. 2K), which were quantified with the assistance of a commercial exosome quantification kit (FIG. 9). Meanwhile, the enzymatic signal generation process was also analysed to maximize the signal-to-noise ratio through modifications of blocking buffer (PBS, PBST, and Block ACE buffer), washing buffer (PBS, PBST, and Block ACE buffer), enzyme concentration (500×, 1000×, and 2000×), the addition of immune affinity enhancers (adding and non-adding), and other variables (FIG. 10A-10E).

After successfully developing the iME beads-based Exo-Aβ42 identification assay, the entire assay was tested for specificity and sensitivity on chip (FIG. 1D). A significant signal was observed upon introducing the targeted molecule Exo-Aβ42, compared to three non-target proteins (Aβ42, CD63, and BSA) (FIG. 2I). It was worth noting that the amyloid beta protein Aβ42 and the exosomal marker protein CD63 did not generate any background noise, indicating that free Aβ42 protein and exosomes do not affect the detection specificity of our assay. These results further confirmed the dual sites targeting mechanism of the iME beads-based Exo-Aβ42 identification assay. After establishing specificity, the sensing behaviour of the assay was investigated by spiking the prepared exosome-amyloid beta associations in PBS and measuring the results on chip (FIGS. 11A-11D). Based on the quantified results, the sensitivity of the platform was accessed with a regression parameter R²=0.987 (FIG. 2M), encompassing the reported concentration range of human plasma amyloid beta42 protein^{51-53.}

### MEMOs Integration and Automation

To achieve fully automated immunosensing operations without requiring user intervention, MEMOs was designed based on a vertical stack assembly including 1) a μ-Exo Biochip embedded with target molecular Exo-Aβ42 identification assay, 2) a μ-Bead EMchip equipped with programmable and addressable motors, and 3) an optical signal sensing model for signal readout (FIG. 1B).

The μ-Exo Biochip consists of reagent reservoirs for encapsulating various assay reagents, an oil zone for reagents isolation and beads/droplets transportation, and a detection chamber for signal readout (FIG. 1D). The structure of the μ-Exo Biochip is laser engraved on a polytetrafluoroethylene (PTFE) substrate due to its excellent resistance to molecular adsorption⁵⁴, which helped prevent assay reagent residues, as well as its non-wetting property (data not shown), which facilitate the manipulation of beads and droplets. Transparent polyethylene terephthalate (PET) sheets were used to create a sealed environment for the assay reagents and to form a transparent detection region for signal readout. Different layers were assembled using patterned double-sided tapes in a convenient and cost-effective manner (FIG. 3A).

To implement the developed iME beads-based Exo-Aβ42 identification assay on the μ-Exo Biochip in a fully automated manner, it was important to thoroughly investigate the key actuation conditions that govern the operation behaviors and movement dynamics of beads and droplets^{44,55,56} The entire system was analyzed with the magnetic force from the magnet motor serving as the external driving force, while the friction force arising from the contact surfaces sliding acted as the opposing external resistance force. The strength of the magnetic force was proportional to the bead loading mass, as the motor-generated magnetic field exerted a direct influence on the bead cluster. The friction force was proportional to the transport velocity of the droplet, which can be regulated by the motor movement speed in the present system.

To differentiate among different motion conditions, the internal force between beads and droplet were considered. The internal force, resulting from the deformation of the droplet surface, was related to the curvature length of the deformed droplet. The maximum internal force can be estimated as the entire circumference of the bead cluster contributes to the deformation of the surface, making it proportional to the cube root of the bead loading mass. In bead operation, the magnetic force served as the driving force, while the internal force acted as a resistance force. In droplet operation, the interaction force of the internal force (with same magnitude but opposite direction of the internal force) serves as the driving force, while the friction force acts as the resistance force. Therefore, to facilitate bead operation the bead loading mass was increased to improve the magnetic force. Alternatively, reducing the actuation velocity can decrease the friction force, thus facilitating more effective droplet operation.

By systematically balancing the three forces present in the present system, a model was simulated to explore the mechanisms underlying the manipulation and interaction between bead and droplet operations (FIG. 3B, line). The simulation results showed three different zones based on different programmed motor speeds and bead loading masses. In the no engagement zone (yellow), the magnetic force was insufficient to overcome either the bead resistance internal force or the droplet friction force, rendering the motor movement ineffective for driving either bead or droplet motions. In the droplet operation zone (pink), as for the bead, the magnetic force was equal to the internal force, while the internal force did not yet reach its maximum at this moment, making it ineffective for bead extraction from the droplet. As for the droplet, the internal force was equal to the friction force, resulting in steady droplet movement. In the bead operation zone (lake blue), as for the bead, the magnetic force exceeded the maximum of the internal force, allowing the beads to be extracted from the droplet. Meanwhile, as for the droplet, the internal force did not exceed the friction force, preventing the motor from operating the droplet. Experiments were also conducted to confirm conclusions drawn from the simulations. The results demonstrated three different operation conditions that aligned with the simulated operation diagram (FIG. 3B, dots). The sequential optical images corresponding to two typical motion zones are shown in FIG. 3C. Under droplet operation conditions (FIG. 3C, bottom), both the beads and the droplet steadily followed the movement of the motor. Under bead operation conditions (FIG. 3C, top), the bead cluster was extracted and consistently followed the actuation of the motor, whereas the droplet was left behind. Based on the simulations and experimental investigations, it was found that adjusting the bead loading mass or regulating the motor speed can effectively switch between bead operation and droplet operation modes, providing flexibility in automating actuation functionalities and biochemical reactions within the system.

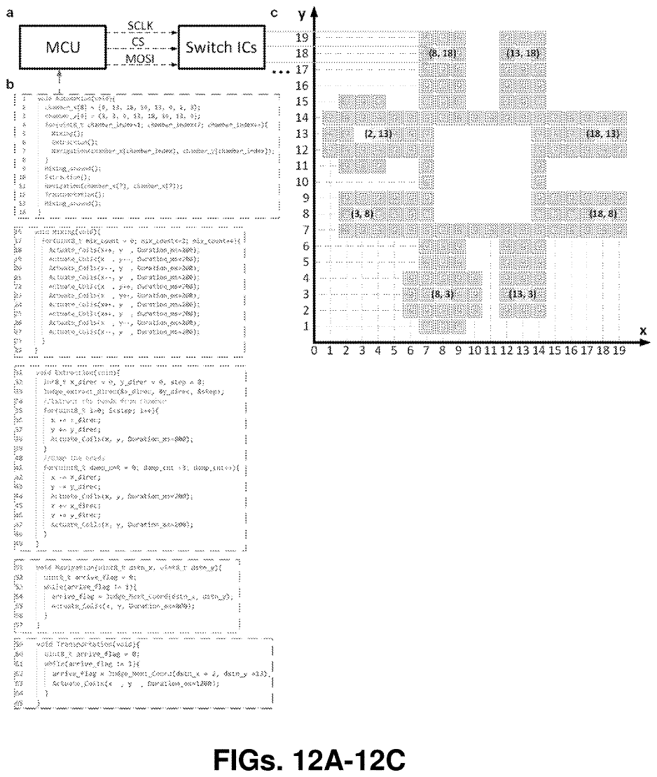

The μ-Bead EMchip contained a programmable navigation matrix including electromagnetic coil elements and magnet motors with the comparable size of the coil unit⁵⁷ (FIG. 1F). The coil elements were programmed and sequentially activated to generate temporary magnetic fields while the current was applied to the adjacent coil elements. Following the guidance of the successively excited magnetic field, the motor served as the driving force for a series of consecutive functionalities (FIG. 1G). To integrate the μ-Bead EMchip with the μ-Exo Biochip, the pattern of the coil elements was designed to correspond to the structure in the microfluidic layer. To achieve full automation of the biological assay, the motor was programmed to follow the predetermined movement commands, facilitating contactless manipulation of beads and droplets to conduct the assay procedure (FIG. 12A-12C).

Facilitated by the integration of the μ-Exo Biochip and μ-Bead EMchip, a showcase of the automated biological assay on chip is shown in FIG. 3D. In the automated testing workflow, the bead and droplet operations were conducted to complete the developed iME beads-based Exo-Aβ42 identification assay on chip. Specifically, after sample loading, a cluster of beads was first magnetically rotated and mixed within the introduced sample droplet to capture exosome with CD63 (FIG. 3D, step 1), providing biomarkers with a means for programmable control and interaction with subsequent reagents. Then the beads were extracted across water-oil interface and transferred to a wash droplet for the removal of excess substances (FIG. 3D, step 2). Subsequently, enzymes conjugated with anti-Aβ42 antibodies were specifically labelled the target molecular Exo-Aβ42 (FIG. 3D, step 3). The next steps in the sequence involved three washings to minimize background interference, followed by the delivery of the labelled molecules to the detection chamber, which contained the substrate reagent to initiate the reaction (FIG. 3D, step 4). Following the redox reaction, a stop buffer droplet was transferred to the detection chamber, where it was merged and mixed with the reactive droplet to stop the reaction and amplify color development, thereby enhancing the signal (FIG. 3D, step 5). To prevent light scattering the magnetic beads from interfering with the sensing accuracy⁴⁴, the beads in the detection chamber were extracted. As demonstrated in the assay and illustrated in FIGS. 3B and 3C, magnetic beads can be extracted from individual droplets or used to transport droplets, depending on the loading mass of the beads and the speed of the magnetic motor. By carefully controlling these parameters, the system facilitates flexible droplet manipulation, allowing either bead-only extraction or bead-assisted droplet transport. In the context of the assay, magnetic beads are first extracted from the sample loading chamber, where they have captured target molecules, thereby facilitating separation of the analyte from the sample droplet. The beads are then sequentially extracted through various processing chambers, including wash chamber 1, the antibody labeling chamber, wash chambers 2, 3, and 4, and ultimately into the detection chamber. The colorimetric readout was then quantified and displayed in a sample-to-answer manner (FIG. 3D, step 6).

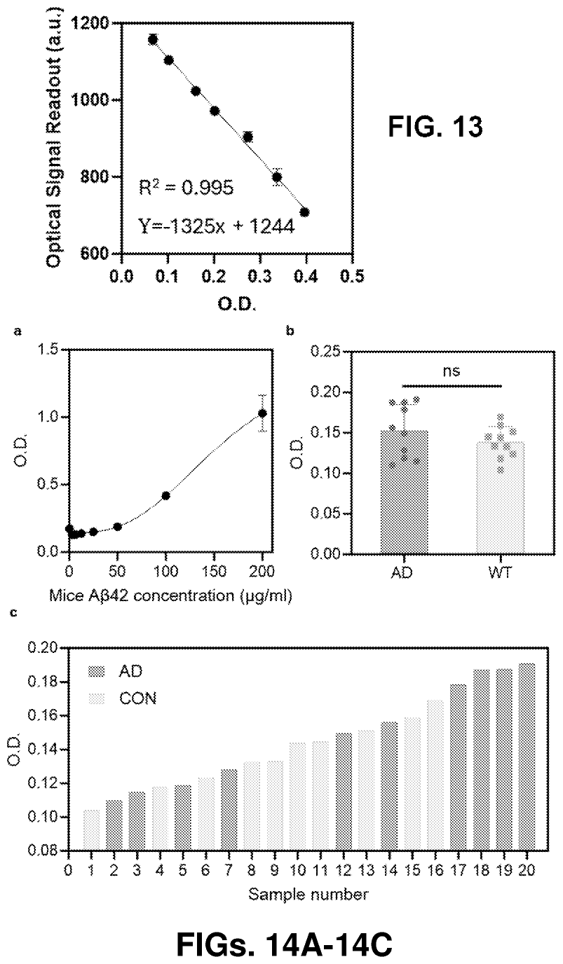

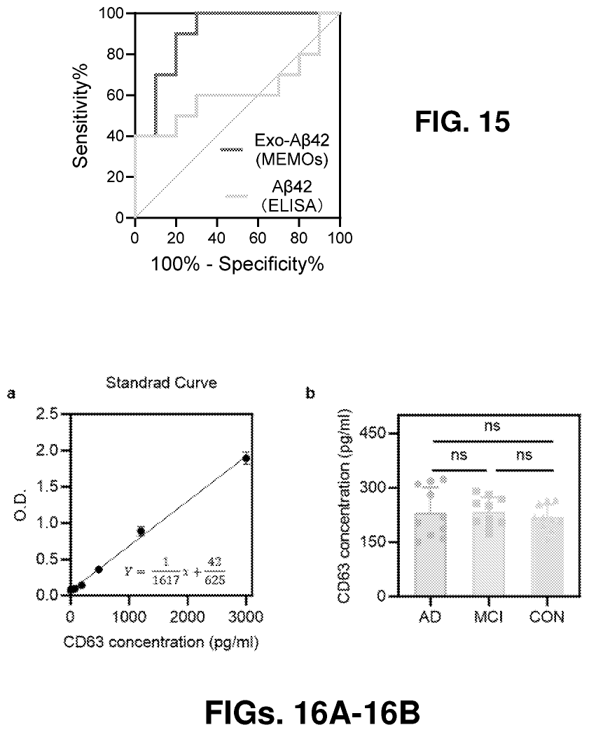

An optical sensor model was integrated directly below the detection chamber to measure light absorbance through the signal readout droplet. The detection accuracy of the optical sensor was adjusted to achieve a linear regression of R²=0.995 compared to a standard microplate reader (FIG. 13). To validate the on-chip assay detection and automation performance of real samples, C57BL/6J and 5×FAD transgenic mice were utilized for blood sampling and on-chip testing (FIG. 3E). The 5×FAD transgenic mouse mode, which exhibit early onset and aggressive age-dependent progression of Aβ aggregation in the brain, is one of the most widely utilized mouse models for Alzheimer's disease research⁵⁸. Immunohistochemical staining of Aβ42 aggregates in the hippocampus of 5×FAD mice was performed to confirm the viability and reliability of the transgenic model (FIG. 3F and data not shown). The immunoreactivity results from all tested 5×FAD mice brains showed robust Aβ42 deposits compared to the C57BL/6J control of the same age. Plasma samples (n=20) were collected and tested on-chip, revealing a significant difference in Exo-Aβ42 level between 5×FAD group and C57BL/6J control (p=0.0014, Mann-Whitney test) (FIGS. 3G and 3H). Additionally, the total Aβ42 protein levels in plasma samples (n=20) using ELISA was measured (FIG. 14A-14C). The results showed that the mean value of total Aβ42 protein level was higher in 5×FAD mice compared to controls. However, this difference was not statistically significant. Receiver operating characteristic (ROC) curve analysis of Exo-Aβ42 levels in plasma measured by MEMOs and Aβ42 levels in plasma tested by ELISA (FIG. 15) indicated the diagnostic potential of MEMOs targeting plasma Exo-Aβ42 biomarker for AD detection, further demonstrating the feasibility of automated capabilities of MEMOs detection.

### MEMOs Clinical Samples Testing Validation

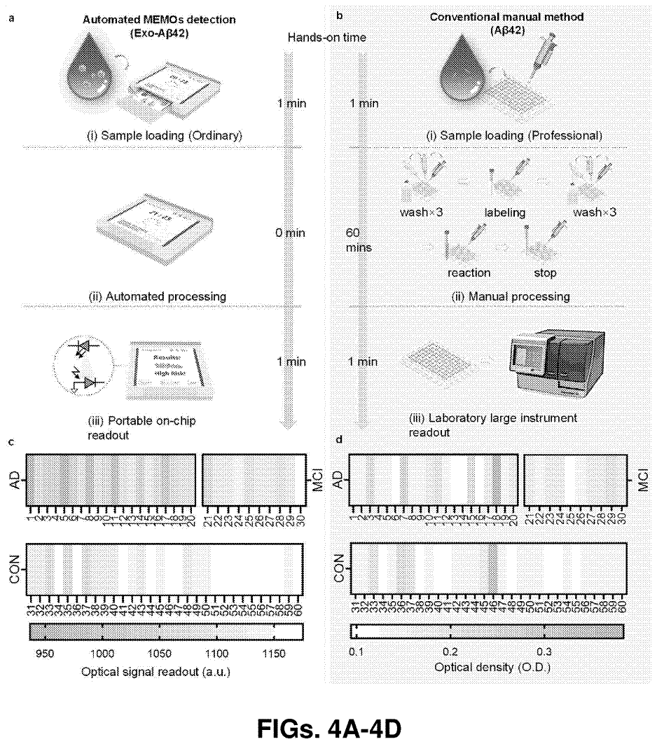

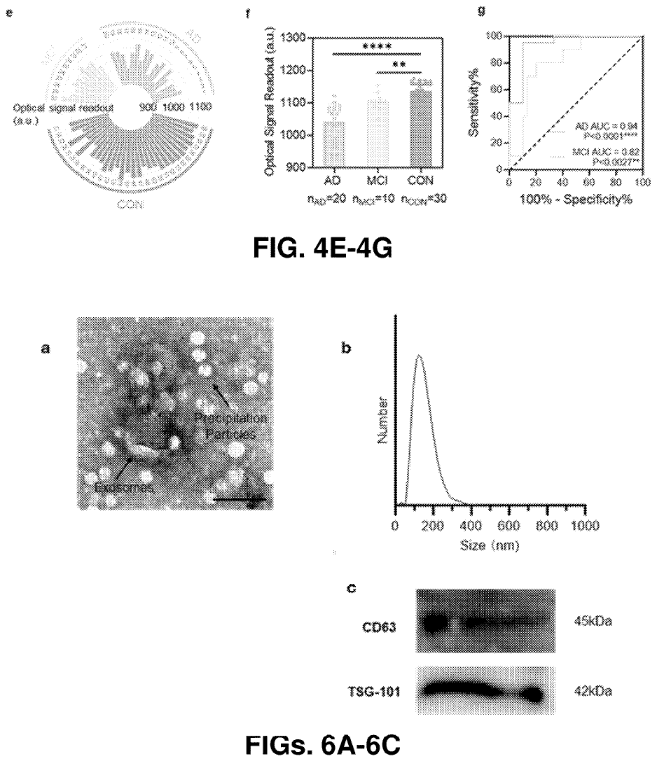

To improve the accessibility of MEMOs in various settings, including hospitals, clinics, and even at home, a user-friendly workflow was adopted, including the following three simple steps (FIG. 4A): (i) Load the collected microliter-volume sample into the μ-Exo Biochip; (ii) Insert the μ-Exo Biochip to initiate the fully automated sample processing and detection procedure; (iii) View the readout signal and interpreted results on the screen or a smart device. Compared to the conventional laboratory-based methods (FIG. 4B), MEMOs provided a user-friendly workflow that did not require specialized training for sample handling. Additionally, MEMOs, with its fully automated sample processing capability, reduced hands-on time from the conventional 60 minutes to 0 minute, eliminating the need for manual pipetting during washing, labelling, reaction, and stopping steps. Moreover, MEMOs offers on-chip readout and signal transmission, overcoming the limitations associated with bulky laboratory signal readers. Therefore, the integration of the μ-Exo Biochip and μ-Bead EMchip enables MEMOs to substantially reduce manual intervention, eliminate complex hands-on processing, and remain feasible under ordinary conditions, thereby improving diagnostic accessibility.

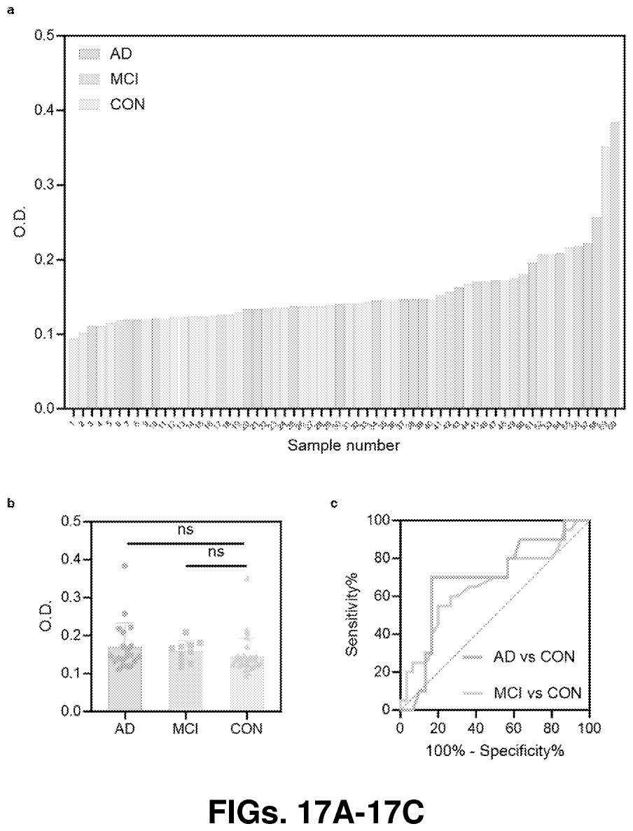

The performance of MEMOs for clinical testing feasibility was investigated using a total of 60 clinical samples, including 20 Alzheimer's samples, 10 mild cognitive impairment samples, and 30 healthy control samples. The optical readouts of the Exo-Aβ42 signal detected by MEMOs are utilized to distinguish Alzheimer's disease (AD), mild cognitive impairment (MCI), and healthy controls (CON) (data not shown). The CD63 levels of clinical samples in each group (n=30) were analyzed and no significant differences was found among the AD, MCI and CON samples (FIGS. 16A and 16B), indicating that the Exo-Aβ42 biomarkers were assessed under the assumption that exosomal CD63 levels do not vary among clinical samples. To evaluate the clinical performance of MEMOs, each plasma specimen was loaded into MEMOs for Exo-Aβ42 detection and simultaneously tested on a microplate for plasma total Aβ42 detection using a commercial ELISA kit for comparison. The sensing signals of Exo-Aβ42 protein detected on-chip for the 60 samples are presented in FIGS. 4C and 4E, while the plasma total Aβ42 detection results are shown in FIG. 4D and FIG. 17A. In the MEMOs Exo-Aβ42 group, it was observed that AD samples exhibited significantly lower readout signals compared to the healthy control group (P<0.0001, Mann-Whitney test), and the differences between MCI and CON were also notable (P<0.0027, Mann-Whitney test) (FIG. 4F). However, the plasma total Aβ level did not show any significant differences among the three groups (FIGS. 17B and 17C). These results indicate a progressively rising trend in plasma Exo-Aβ42 levels across the AD, MCI and CON stages of Alzheimer's disease, suggesting the potential of plasma Exo-Aβ42 as a predictor for differentiating different stages of Alzheimer's disease progression.

ROC analysis was also conducted to evaluate the diagnostic potential of the present platform for AD and to determine the critical value of Exo-Aβ42 protein marker among the AD, MCI and CON groups (FIG. 4G). The area under the curve (AUC) value was found to be 0.94, with a P value of less than 0.0001 for the comparison between AD and CON groups, indicating excellent diagnostic accuracy (AUC≥0.9) for AD, based on the established guidelines for AUC interpretation⁵⁹. Additionally, the AUC value is 0.82, with a P value of less than 0.01 for the MCI vs CON groups, which was interpreted as demonstrating good and considerable testing performance, further suggesting capability of the platform for monitoring AD progression. The ROC curve analysis indicated that present platform can diagnose AD patient groups with a sensitivity of 95% and specificity of 90% (cut-off value≤1104) and can distinguish MCI patient groups from healthy CON groups with a sensitivity of 80% and specificity of 80% (cut-off value≤1115). These results demonstrated the potential of MEMOs to automate Alzheimer's disease blood testing and suggest that it provides an accessible and convenient pathway towards diagnosing and monitoring the progression of the disease.

### Discussion

In this study, a fully integrated and automated system was introduced, MEMOs, for the detection of the AD blood biomarker, Exo-Aβ42 protein. MEMOs autonomously handled biomarker processing, analysis, and data transmission through a programmable actuation system, hence eliminating the need for manual intervention. Unlike previous technologies for the monitoring of AD biomarkers, which generally operate within conventional laboratory environments, MEMOs offer a more accessible solution for a wider range of settings. This allows non-specialists to conduct tests at home, while still maintaining high sensitivity and specificity in diagnosing Alzheimer's disease. The benefits of MEMOs stem from a comprehensive combination of the following components: 1) a μ-Exo Biochip, encapsulated within a magnetic field-responsive assay, specially designed to target dual marker sites of the Exo-Aβ42 protein, allowing for detection limits as low as several picograms per milliliter; 2) a μ-Bead EMchip, featuring a programmable navigation matrix, engineered for robustly addressable magnetic motor actuation in accordance with predetermined commands; and 3) the integrated system facilitates contactless control of both bead and droplet movements, facilitating diverse functionalities and the execution of immunosensing operations in a fully automated manner.

Utilizing MEMOs, the levels of blood Exo-Aβ42 in both animal models (including healthy and AD transgenic mice) and clinical samples (plasma from healthy individuals, patients with mild cognitive impairment, and Alzheimer's patients) were assessed. The findings revealed significant correlations between blood Exo-Aβ42 levels and the progressive stages of AD, underscoring the potential of MEMOs as an accessible point-of-care diagnostic device for monitoring the development of AD. Currently, this system offers a convenient method for evaluating of blood Exo-Aβ42 levels. Despite the pressing need for blood testing for AD, such assessment has not been widely implemented in routine clinical practice, primarily due to lack of established protocols rather than lack of need^{23,25,26,28,60}. As a consequence, MEMOs can serve as a practical instrument for providing clinical insights during early medical assessments to alleviate the pressure on healthcare systems and facilitating the rational allocation of medical resources. Furthermore, the results obtained from MEMOs can be interpreted in conjunction with other diagnostic modalities, such as cerebrospinal fluid analysis and positron emission tomography, to improve clinical decision-making.

On a broader level, the versatility of MEMOs can be improved to accommodate large-scale, high-throughput, and multiplexed biomarker detection. From a systems perspective, the current configuration is designed to execute an iME beads-based assay for Exo-Aβ42 identification. The arrangement of patterns in the μ-Exo Biochip and the navigation matrix in the μ-Bead EMchip can be easily modified to adapt to different assay protocols. Additionally, the integrated readout module currently relies on a colorimetric measurement sensor. Future improvements could include the automation and integration of other optical and electrochemical signal readout models to meet diverse signal generation requirements. Based on the configurable assay actuation patterns and signal acquisition approaches, MEMOs can theoretically support multiple parallel detection channels, enabling large-scale high-throughput screening. From an application perspective, MEMOs can be customized for on-demand analysis of other disease-associated exosome and/or protein biomarkers. Furthermore, leveraging the technological capabilities for large-scale and high-throughput actuation, MEMOs has the potential for simultaneous analysis of multiple targets, thereby improving healthcare efficiency. The prospective incorporation of artificial intelligent algorithms can further streamline the operational steps required for multiplexed testing, facilitating more intricate bioanalytical operations.

### References

1. World Health Organization. Ageing and health. https://www.who.int/news-room/fact-sheets/detail/ageing-and-health (2024).
2. Khan, H. T. A., Addo, K. M. & Findlay, H. Public Health Challenges and Responses to the Growing Ageing Populations. Public Health Challenges 3, e213 (2024).
3. 2024 Alzheimer's disease facts and figures. Alzheimer's & Dementia 20, 3708-3821 (2024).
4. National Institute on Aging. Alzheimer's Disease Fact Sheet (2023). https://www.nia.nih.gov/health/alzheimers-and-dementia/alzheimers-disease-fact-sheet.
5. Zhang, J. et al. Recent advances in Alzheimer's disease: mechanisms, clinical trials and new drug development strategies. Signal Transduction and Targeted Therapy 2024 9:1 9, 1-35 (2024).
6. Li, X. et al. Global, regional, and national burden of Alzheimer's disease and other dementias, 1990-2019. Front Aging Neurosci 14, 937486 (2022).
7. Culberson, J. W., Kopel, J., Sehar, U. & Reddy, P. H. Urgent needs of caregiving in ageing populations with Alzheimer's disease and other chronic conditions: Support our loved ones. Ageing Res Rev 90, 102001 (2023).
8. Chen, S. et al. The global macroeconomic burden of Alzheimer's disease and other dementias: estimates and projections for 152 countries or territories. Lancet Glob Health 12, e1534-e1543 (2024).
9. Rasmussen, J. & Langerman, H. Alzheimer's Disease—Why We Need Early Diagnosis. Degener Neurol Neuromuscul Dis 9, 123 (2019).
10. How diagnostic tools can help reshape Alzheimer's care. https://www.nature.com/articles/d42473-023-00325-z (2024).
11. Wójcik, D., Szczechowiak, K., Zboch, M. & Pikala, M. Effectiveness of the Open Screening Programs in Recruiting Subjects to Prodromal and Mild Alzheimer's Disease Clinical Trials. J Prev Alzheimers Dis 7, 251-255 (2020).
12. Leuzy, A. et al. REAL AD-Validation of a realistic screening approach for early Alzheimer's disease. Alzheimers Dement 20, (2024).
13. Chapman, R. M. et al. Diagnosis of Alzheimer's disease using neuropsychological testing improved by multivariate analyses. J Clin Exp Neuropsychol 32, 793-808 (2010).
14. Donders, J. The incremental value of neuropsychological assessment: A critical review. Clin Neuropsychol 34, 56-87 (2020).
15. Watt, S. & Crowe, S. F. Examining the beneficial effect of neuropsychological assessment on adult patient outcomes: a systematic review. Clin Neuropsychol 32, 368-390 (2018).
16. Chouliaras, L. & O'Brien, J. T. The use of neuroimaging techniques in the early and differential diagnosis of dementia. Molecular Psychiatry 2023 28:10 28, 4084-4097 (2023).
17. Haller, S., Jäger, H. R., Vernooij, M. W. & Barkhof, F. Neuroimaging in Dementia: More than Typical Alzheimer Disease. Radiology 308, (2023).
18. Kim, J., Jeong, M., Stiles, W. R. & Choi, H. S. Neuroimaging Modalities in Alzheimer's Disease: Diagnosis and Clinical Features. Int J Mol Sci 23, 6079 (2022).
19. Roebuck-Spencer, T. M. et al. Cognitive Screening Tests Versus Comprehensive Neuropsychological Test Batteries: A National Academy of Neuropsychology Education Paper. Archives of Clinical Neuropsychology 32, 491-498 (2017).
20. Howieson, D. Current limitations of neuropsychological tests and assessment procedures. Clin Neuropsychol 33, 200-208 (2019).
21. Schroeder, R. W., Martin, P. K. & Walling, A. Neuropsychological Evaluations in Adults. Am Fam Physician 99, 101-108 (2019).
22. Linden, D. E. J. The Challenges and Promise of Neuroimaging in Psychiatry. Neuron 73, 8-22 (2012).
23. Barthélemy, N. R. et al. Highly accurate blood test for Alzheimer's disease is similar or superior to clinical cerebrospinal fluid tests. Nature Medicine 2024 30:4 30, 1085-1095 (2024).
24. Paterson, R. W. et al. Cerebrospinal fluid in the differential diagnosis of Alzheimer's disease: Clinical utility of an extended panel of biomarkers in a specialist cognitive clinic. Alzheimers Res Ther 10, 1-11 (2018).
25. Palmqvist, S. et al. Blood Biomarkers to Detect Alzheimer Disease in Primary Care and Secondary Care. JAMA 332, 1245-1257 (2024).
26. Kiddle, S. J., Voyle, N. & Dobson, R. J. B. A blood test for Alzheimer's disease: progress, challenges and recommendations. J Alzheimers Dis 64, S289 (2018).
27. Blennow, K. Phenotyping Alzheimer's disease with blood tests. Science (1979) 373, 626-628 (2021).
28. Blood tests for Alzheimer's disease in the era of anti-amyloid therapies. https://www.nature.com/articles/d42473-024-00277-y.
29. Brand, A. L. et al. The performance of plasma amyloid beta measurements in identifying amyloid plaques in Alzheimer's disease: a literature review. Alzheimers Res Ther 14, 195 (2022).
30. Lövheim, H. et al. Plasma concentrations of free amyloid β cannot predict the development of Alzheimer's disease. Alzheimer's & Dementia 13, 778-782 (2017).
31. Wang, J., Gu, B. J., Masters, C. L. & Wang, Y. J. A systemic view of Alzheimer disease—insights from amyloid-β metabolism beyond the brain. Nature Reviews Neurology 2017 13:10 13, 612-623 (2017).
32. Wisch, J. K. et al. Predicting continuous amyloid PET values with CSF and plasma Aβ42/Aβ40. Alzheimer's & Dementia: Diagnosis, Assessment & Disease Monitoring 15, e12405 (2023).
33. De Rojas, I. et al. Correlations between plasma and PET beta-amyloid levels in individuals with subjective cognitive decline: The Fundació ACE Healthy Brain Initiative (FACEHBI). Alzheimers Res Ther 10, 1-12 (2018).
34. Hampel, H. et al. Blood-based biomarkers for Alzheimer's disease: Current state and future use in a transformed global healthcare landscape. Neuron 111, 2781 (2023).
35. Kourtis, L. C., Regele, O. B., Wright, J. M. & Jones, G. B. Digital biomarkers for Alzheimer's disease: the mobile/wearable devices opportunity. npj Digital Medicine 2019 2:1 2, 1-9 (2019).
36. Hosseini, S., Vázquez-Villegas, P., Rito-Palomares, M. & Martinez-Chapa, S. O. Advantages, Disadvantages and Modifications of Conventional ELISA. SpringerBriefs in Applied Sciences and Technology 67-115 (2018) doi:10.1007/978-981-10-6766-2_5.
37. Li, J. et al. Nanosensor-Driven Detection of Neuron-Derived Exosomal Aβ42 with Graphene Electrolyte-Gated Transistor for Alzheimer's Disease Diagnosis. Anal Chem 95, 5719-5728 (2023).
38. Wang, L., Chen, H., Ma, S., Chang, M. & Zhang, X. Ultra-sensitive SERS detection of Aβ 1-42 for Alzheimer's disease using graphene oxide/gold nanohybrids. Vib Spectrosc 129, 103614 (2023).
39. Zu, L. et al. Ultrasensitive and Multiple Biomarker Discrimination for Alzheimer's Disease via Plasmonic & Microfluidic Sensing Technologies. Advanced Science 11, 2308783 (2024).
40. Lim, C. Z. J. et al. Subtyping of circulating exosome-bound amyloid β reflects brain plaque deposition. Nature Communications 2019 10:1 10, 1-11 (2019).
41. Sun, M. & Chen, Z. Unveiling the Complex Role of Exosomes in Alzheimer's Disease. J Inflamm Res 17, 3921 (2024).
42. Sardar Sinha, M. et al. Alzheimer's disease pathology propagation by exosomes containing toxic amyloid-beta oligomers. Acta Neuropathol 136, 41 (2018).
43. Rajendran, L. et al. Alzheimer's disease β-amyloid peptides are released in association with exosomes. Proc Natl Acad Sci U S A 103, 11172-11177 (2006).
44. Cai, G. et al. Magnetic Bead Manipulation in Microfluidic Chips for Biological Application. Cyborg and Bionic Systems 4, (2023).
45. Hu, X., Gao, X., Chen, S., Guo, J. & Zhang, Y. DropLab: an automated magnetic digital microfluidic platform for sample-to-answer point-of-care testing—development and application to quantitative immunodiagnostics. Microsystems & Nanoengineering 2023 9:1 9, 1-12 (2023).
46. He, L. et al. Digital immunoassay for biomarker concentration quantification using solid-state nanopores. Nature Communications 2021 12:1 12, 1-11 (2021).
47. Lee, S. et al. Amplified fluorogenic immunoassay for early diagnosis and monitoring of Alzheimer's disease from tear fluid. Nature Communications 2023 14:1 14, 1-15 (2023).
48. Yousif, G. et al. Exosomes Derived Neuronal Markers: Immunoaffinity Isolation and Characterization. Neuromolecular Med 24, 339-351 (2021).
49. Hu, S. et al. Sensitive detection of multiple blood biomarkers via immunomagnetic exosomal PCR for the diagnosis of Alzheimer's disease. Sci Adv 10, 3088 (2024).
50. Cetin, S., Knez, D., Gobec, S., Kos, J. & Pišlar, A. Cell models for Alzheimer's and Parkinson's disease: At the interface of biology and drug discovery. Biomedicine & Pharmacotherapy 149, 112924 (2022).
51. Feinkohl, I. et al. Plasma Amyloid Concentration in Alzheimer's Disease: Performance of a High-Throughput Amyloid Assay in Distinguishing Alzheimer's Disease Cases from Controls. Journal of Alzheimer's Disease 74, 1285 (2020).
52. Janelidze, S. et al. Plasma β-amyloid in Alzheimer's disease and vascular disease. Scientific Reports 2016 6:1 6, 1-11 (2016).
53. de Kort, A. M. et al. Plasma amyloid beta 42 is a biomarker for patients with hereditary, but not sporadic, cerebral amyloid angiopathy. Alzheimers Res Ther 15, 1-14 (2023).
54. Dhanumalayan, E. & Joshi, G. M. Performance properties and applications of polytetrafluoroethylene (PTFE)—a review. Advanced Composites and Hybrid Materials 2018 1:2 1, 247-268 (2018).
55. Long, Z., Shetty, A. M., Solomon, M. J. & Larson, R. G. Fundamentals of magnet-actuated droplet manipulation on an open hydrophobic surface. Lab Chip 9, 1567-1575 (2009).
56. Kong, T., Brien, R., Njus, Z., Kalwa, U. & Pandey, S. Motorized actuation system to perform droplet operations on printed plastic sheets. Lab Chip 16, 1861-1872 (2016).
57. Yu, W. et al. A ferrobotic system for automated microfluidic logistics. Sci Robot 5, (2020).
58. Zhong, M. Z., Peng, T., Duarte, M. L., Wang, M. & Cai, D. Updates on mouse models of Alzheimer's disease. Molecular Neurodegeneration 2024 19:1 19, 1-33 (2024).
59. Nahm, F. S. Receiver operating characteristic curve: overview and practical use for clinicians. Korean J Anesthesiol 75, 25-36 (2022).
60. Schindler, S. E. et al. Acceptable performance of blood biomarker tests of amyloid pathology—recommendations from the Global CEO Initiative on Alzheimer's Disease. Nature Reviews Neurology 2024 20:7 20, 426-439 (2024).

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Those skilled in the art will recognize, or be able to ascertain, using no more than routine experimentation, many equivalents to the specific embodiments of the method and compositions described herein. Such equivalents are intended to be encompassed by the following claims.

We claim:

1. A microfluidic device for automated analyte detection comprising:
(a) a biochip;
(b) an electromagnetic chip comprising a programmable navigation matrix;
wherein the biochip and the electromagnetic chip are oriented parallel to each other, and
wherein the electromagnetic chip is directly below the biochip.

2. The microfluidic device of claim 1, wherein the biochip comprises:
(i) a sample loading chamber;
(ii) one or more wash chambers;
(iii) a binding chamber; and
(iv) a detection chamber;
wherein the sample loading chamber, one or more wash chambers, the binding chamber, and the detection chamber are in fluid communication with each other.

3. The microfluidic device of claim 1, further comprising a plurality of immunomagnetic exosome beads; optionally wherein the plurality of immunomagnetic exosome beads is provided as a separate component or is pre-loaded in the sample loading chamber.

4. The microfluidic device of claim 1, wherein the biochip comprises two or more wash chambers,
wherein a first wash chamber is in direct fluid communication with the sample loading chamber and the binding chamber, and
wherein a second wash chamber is in direct fluid communication with the detection chamber and the binding chamber.

5. The microfluidic device of claim 2, wherein the biochip further comprises a stop buffer chamber, and wherein the detection chamber is in direct fluid communication with the stop buffer chamber.

6. The microfluidic device of claim 1, wherein the electromagnetic chip comprises a programmable navigation matrix comprising:
(1) a plurality of electromagnetic coils,
(2) one or more switch integrated circuits electrically connected to the plurality of electromagnetic coils,
(3) one or more magnetic motors, and
(4) a microcontroller configured to regulate the activation of electromagnetic coils for electromagnetic actuation of the magnetic motor.

7. The microfluidic device of claim 6, wherein the plurality of electromagnetic coils is arranged such that they align with the sample loading chamber, the one or more wash chambers, the binding chamber, and the detection chamber.

8. The microfluidic device of claim 6, the plurality of electromagnetic coils is arranged such that they align with the stop buffer chamber.

9. The microfluidic device of claim 1, further comprising a material exchange area,
wherein the material exchange area is in fluid connection with the sample loading chamber, the one or more wash chambers, the binding chamber, and the detection chamber, and
optionally wherein the material exchange region is centrally positioned on the biochip.

10. The microfluidic device of claim 2, wherein each of the sample loading chamber, the one or more wash chambers; the binding chamber, and the detection chamber comprise a mixing area and a flow region.

11. The microfluidic device of claim 1, further comprising:
(c) an optical sensing module; and
(d) optionally an operation module.

12. The microfluidic device of claim 11, wherein the optical sensing module comprises:
(i) a light source aligned with the detection chamber to emit light through the signal readout droplet;
(ii) a photodetector configured to receive transmitted or absorbed light from a droplet in the detection chamber and generate an output signal corresponding to the transmitted or absorbed light levels; and
(iii) optionally, a supporting structure to secure the optical sensing module in fixed alignment with the detection chamber.

13. The microfluidic device of claim 11, wherein the light source of the optical sensing module is aligned below the detection chamber.

14. The microfluidic device of claim 1, wherein the biochip comprises
(a) one or more barrier layers, comprising a top layer and a bottom layer;
(b) one or more chamber layers; and
(c) optionally one or more adhesive layers,
wherein when the biochip is assembled the one or more chamber layers form one or more chambers and one or more flow paths,
and the one or more chamber layers and one or more adhesive layers are positioned between the top layer and the bottom layer.

15. The microfluidic device of claim 14, wherein the one or more barrier layers are formed of one or more polymers selected from the group consisting of polyethylene terephthalate, polycarbonate, cyclic olefin copolymer, cyclic olefin polymer, poly(methyl methacrylate), polyethylene naphthalate (PEN), clarified polypropylene, fluoropolymers such as Cytop, cellulose acetate Films and thermoplastic polyurethane.

16. The microfluidic device of claim 14, wherein the one or more chamber layers are formed from one or more polymers selected from Polytetrafluoroethylene, DMS, cyclic olefin copolymer, cyclic olefin polymer, poly(methyl methacrylate), polycarbonate, clarified polypropylene, Fluorinated Ethylene Propylene, Polyethylene Naphthalate (PEN), and TPU.

17. A method for detecting an analyte in a biological sample comprising:
(a) loading the biological sample into the sample loading chamber of the biochip of the microfluidic device of claim 1; and
(b) initiating the generation of an electromagnetic field.

18. The method of claim 17, wherein:
the sample loading chamber of the biochip comprises a plurality of immunomagnetic exosome beads pre-loaded therein; or
a plurality of immunomagnetic exosome beads is provided as a separate component and the method further comprises loading the plurality of immunomagnetic exosome beads into the biochip prior to or in step (a).

19. The method of claim 18, wherein the plurality of immunomagnetic exosome beads is functionalized with a binding agent that specifically binds one or more exosomal markers.

20. The method of claim 19, wherein the exosomal marker is selected from the group consisting of CD63, CD9, CD81, CD82, CD151, Anti-HSP70, anti-HSP90, or Annexin V Tim4 protein, or a combination thereof.
